(19) United States
(12) Patent Application Publication
(10) Pub. No.: US 2026/0354900 A1
(43) Pub. Date: Nov. 12, 2026

(54) SYSTEM AND METHOD FOR GUIDING TRANS-CATHETER AORTIC VALVE REPLACEMENT

(71) Applicant: Libra Science Ltd., Road Town, Tortola (VG)

(72) Inventors: Shlomo BEN-HAIM, Milan (IT); Barouch Asi ELAD, Kiryat Ono (IL)

(73) Assignee: Libra Science Ltd., Road Town, Tortola (VG)

(21) Appl. No.: 19/135,118

(22) PCT Filed: Dec. 4, 2023
(86) PCT No.: PCT/IB2023/062176

§ 371 Date: Jun. 3, 2025

Related U.S. Application Data
(60) Provisional application No. 63/430,063, filed on Dec. 5, 2022.
(60) Provisional application No. 63/466,722, filed on May 16, 2023.
(60) Provisional application No. 63/528,450, filed on Jul. 24, 2023.
(60) Provisional application No. 63/530,724, filed on Aug. 4, 2023.
(60) Provisional application No. 63/531,063, filed on Aug. 7, 2023.

(51) Int. Cl. A61B 34/10; A61B 34/00; A61B 90/00; G06T 7/00; G06T 7/10; G06T 7/60; G06T 11/60

(52) U.S. Cl. CPC A61B 34/10; A61B 34/25; A61B 90/37; G06T 7/0012; G06T 7/10; G06T 7/60; G06T 11/60; A61B 2034/107; A61B 2090/376; G06T 2207/10121; G06T 2207/20081; G06T 2207/30048; G06T 2207/30052

(57) ABSTRACT

There is provided a computer implemented method of computing curvature of a guidewire depicted in a medical image, comprising: accessing at least one fluoroscopic image depicting the guidewire, analyzing the at least one fluoroscopic image to identify the guidewire with local curvature, and computing a local curvature of the guidewire depicted in the at least one fluoroscopic image.

There is provided a computer implemented method of guiding a trans-catheter aortic valve replacement intervention in a subject, comprising: accessing at least one fluoroscopic image depicting an aortic valve prosthesis device in an aorta of the subject, analyzing the at least one fluoroscopic image for determining the aortic valve prosthesis device and an anatomical landmark, and computing a depth and/or a tilt of the aortic valve prosthesis device relative to the anatomical landmark. Alternatively or additionally, the method includes computing a width of the aortic valve prosthesis device.

### RELATED APPLICATIONS

This application claims the benefit of priority of U.S. Provisional Patent Application No. 63/430,063 filed on Dec. 5, 2022, U.S. Provisional Patent Application No. 63/466,722 filed on May 16, 2023, U.S. Provisional Patent Application No. 63/528,450 filed on Jul. 24, 2023, U.S. Provisional Patent Application No. 63/530,724 filed on Aug. 4, 2023, and U.S. Provisional Patent Application No. 63/531,063 filed on Aug. 7, 2023, the contents of which are incorporated herein by reference in their entirety.

### BACKGROUND

The present invention, in some embodiments thereof, relates to aortic valve replacement and, more specifically, but not exclusively, to systems and methods for guiding aortic valve replacement.

Currently, implantable medical devices, such as large stents, scaffolds, and other cardiac intervention devices are utilized to repair or replace problem native biological systems. For example, heart valve replacement in patients with severe valve disease is a common surgical procedure. The replacement can conventionally be performed by open heart surgery, in which the heart is usually arrested and the patient is placed on a heart bypass machine. In recent years, prosthetic heart valves have been developed which are implanted using minimally invasive procedures such as transapical or percutaneous approaches. These procedures involve compressing the prosthetic heart valve radially to reduce its diameter, inserting the prosthetic heart valve into a delivery device, such as a catheter, and advancing the delivery device to the correct anatomical position in the heart. Once properly positioned, the prosthetic heart valve is deployed by radial expansion within the native valve annulus.

### SUMMARY

According to some embodiments of the present invention, there is provided a computer implemented method of computing curvature of a guidewire depicted in a medical image, comprising: accessing at least one fluoroscopic image depicting the guidewire, analyzing the at least one fluoroscopic image to identify the guidewire with local curvature, and computing a local curvature of the guidewire depicted in the at least one fluoroscopic image.

Optionally, presenting comprises generating an overlay over a portion of the at least one fluoroscopic image indicating where the local curvature is above a threshold.

Optionally, the overlay is coded according to an amount of the local radius of the local curvature.

Optionally, the overlay is color coded for indicating a range of a plurality of ranges of the radius of the local curvature with a different color for each range.

Optionally, further comprising measuring the local curvature at a plurality of regions along a length of the guidewire to obtain a plurality of curvatures.

Optionally, the overlay visually indicates the plurality of curvatures along the length of the guidewire according to a visual coding.

Optionally, computing the local curvature comprises computing a local radius of the local curvature.

Optionally, the local radius is computed by fitting a circle to the local curvature and measuring a radius of the circle.

Optionally, analyzing comprises feeding the at least one fluoroscopic image into a machine learning model trained for segmenting the guidewire within the at least one fluoroscopic image.

Optionally, the machine learning model is trained on a training dataset of a plurality of sample fluoroscopic images depicting sample guidewires with different amounts of curvature, labelled with a ground truth segmentation of the sample guidewire.

Optionally, further comprising calculating the local curvature relative to a threshold indicating high curvature likely to lead to an adverse event, and presenting the indication when the local radius is below the threshold.

Optionally, the guidewire comprises a stiff guidewire.

Optionally, further comprising computing a maximal curvature of the guidewire depicted in the at least one fluoroscopic image, and presenting the maximal curvature on the display.

Optionally, further comprising determining whether the local curvature of the guidewire is within pre-defined thresholds, and presenting the indication when the local curvature is not within the pre-defined thresholds.

Optionally, further comprising monitoring a plurality of fluoroscopic images, determining a maximal curvature distance of the guidewire from a native aortic annulus plane according to the monitoring, and presenting the maximal curvature distance.

Optionally, further comprising determining whether the local curvature of the guidewire is above the annulus of a subject, and presenting an indication of whether the local curvature is above the annulus of the subject.

Optionally, further comprising presenting an indication of the local curvature on a display.

Optionally, the accessing, the analysing, and the computing are performed during a transcatheter aortic valve implantation being performed on a subject, and the guidewire is located across and/or in proximity to a native aortic annulus of the subject.

According to some embodiments of the present invention, there is provided an apparatus for computing curvature of a guidewire depicted in a medical image, comprising: at least one processor executing a code for: accessing at least one fluoroscopic image depicting the guidewire, analyzing the at least one fluoroscopic image to identify the guidewire with local curvature, and computing a local curvature of the guidewire depicted in the at least one fluoroscopic image.

Optionally, presenting comprises generating an overlay over a portion of the at least one fluoroscopic image indicating where the local curvature is above a threshold.

Optionally, the overlay is coded according to an amount of the local radius of the local curvature.

Optionally, the overlay is color coded for indicating a range of a plurality of ranges of the radius of the local curvature with a different color for each range.

Optionally, further comprising code for measuring the local curvature at a plurality of regions along a length of the guidewire to obtain a plurality of curvatures.

Optionally, the overlay visually indicates the plurality of curvatures along the length of the guidewire according to a visual coding.

Optionally, computing the local curvature comprises computing a local radius of the local curvature.

Optionally, the local radius is computed by fitting a circle to the local curvature and measuring a radius of the circle.

Optionally, analyzing comprises feeding the at least one fluoroscopic image into a machine learning model trained for segmenting the guidewire within the at least one fluoroscopic image.

Optionally, the machine learning model is trained on a training dataset of a plurality of sample fluoroscopic images depicting sample guidewires with different amounts of curvature, labelled with a ground truth segmentation of the sample guidewire.

Optionally, further comprising code for calculating the local curvature relative to a threshold indicating high curvature likely to lead to an adverse event, and presenting the indication when the local radius is below the threshold.

Optionally, the guidewire comprises a stiff guidewire.

Optionally, further comprising code for computing a maximal curvature of the guidewire depicted in the at least one fluoroscopic image, and presenting the maximal curvature on the display.

Optionally, further comprising code for determining whether the local curvature of the guidewire is within pre-defined thresholds, and presenting the indication when the local curvature is not within the pre-defined thresholds.

Optionally, further comprising code for monitoring a plurality of fluoroscopic images, determining a maximal curvature distance of the guidewire from a native aortic annulus plane according to the monitoring, and presenting the maximal curvature distance.

Optionally, further comprising code for determining whether the local curvature of the guidewire is above the annulus of a subject, and presenting an indication of whether the local curvature is above the annulus of the subject.

Optionally, further comprising code for presenting an indication of the local curvature on a display.

Optionally, the accessing, the analysing, and the computing are performed during a transcatheter aortic valve implantation being performed on a subject, and the guidewire is located across and/or in proximity to a native aortic annulus of the subject.

According to some embodiments of the present invention, there is provided a computer implemented method of guiding a trans-catheter aortic valve replacement intervention in a subject, comprising: accessing at least one fluoroscopic image depicting an aortic valve prosthesis device in an aorta of the subject, analyzing the at least one fluoroscopic image to identify the aortic valve prosthesis device in the aorta, and computing a width of the aortic valve prosthesis device in the aorta.

Optionally, further comprising generating an overlay over the at least one fluoroscopic image indicating where the width of the aortic valve prosthesis device is measured.

Optionally, the width is of a distal end of the aortic valve prosthesis device in a compressed state and/or partially expanded state.

Optionally, analyzing comprises feeding the at least one fluoroscopic image into a machine learning model trained for segmenting the aortic valve prosthesis device within the at least one fluoroscopic image.

Optionally, analyzing comprises feeding the at least one fluoroscopic image into a machine learning model trained for segmenting the aortic valve prosthesis device within the at least one fluoroscopic image, and wherein the width is computed by measuring an end of the segmented aortic valve prosthesis device.

Optionally, the machine learning model is trained on a training dataset of a plurality of sample fluoroscopic images depicting sample aortic valve prosthesis devices, labelled with a ground truth segmentation of the sample aortic valve prosthesis device.

Optionally, further comprising tracking the width over a plurality of fluoroscopic images, determining a maximal width, and presenting the maximal width on the display.

Optionally, further comprising computing a length of the aortic valve prosthesis device, tracking the length as the width changes, and determining a maximal length of the aortic valve prosthesis device.

Optionally, further comprising presenting the width on a display.

According to some embodiments of the present invention, there is provided an apparatus for guiding a trans-catheter aortic valve replacement intervention in a subject, comprising: at least one processor executing a code for: accessing at least one fluoroscopic image depicting an aortic valve prosthesis device in an aorta of the subject, analyzing the at least one fluoroscopic image to identify the aortic valve prosthesis device in the aorta, and computing a width of the aortic valve prosthesis device in the aorta.

Optionally, further comprising code for generating an overlay over the at least one fluoroscopic image indicating where the width of the aortic valve prosthesis device is measured.

Optionally, the width is of a distal end of the aortic valve prosthesis device in a compressed state and/or partially expanded state.

Optionally, analyzing comprises feeding the at least one fluoroscopic image into a machine learning model trained for segmenting the aortic valve prosthesis device within the at least one fluoroscopic image.

Optionally, analyzing comprises feeding the at least one fluoroscopic image into a machine learning model trained for segmenting the aortic valve prosthesis device within the at least one fluoroscopic image, and wherein the width is computed by measuring an end of the segmented aortic valve prosthesis device.

Optionally, the machine learning model is trained on a training dataset of a plurality of sample fluoroscopic images depicting sample aortic valve prosthesis devices, labelled with a ground truth segmentation of the sample aortic valve prosthesis device.

Optionally, further comprising code for tracking the width over a plurality of fluoroscopic images, determining a maximal width, and presenting the maximal width on the display.

Optionally, further comprising code for computing a length of the aortic valve prosthesis device, tracking the length as the width changes, and determining a maximal length of the aortic valve prosthesis device.

Optionally, further comprising code for presenting the width on a display.

According to some embodiments of the present invention, there is provided a computer implemented method of guiding a trans-catheter aortic valve replacement intervention in a subject, comprising: accessing at least one fluoroscopic image depicting an aortic valve prosthesis device in an aorta of the subject, the at least one fluoroscopic image excludes injected contrast, analyzing the at least one fluoroscopic image to identify a distal end of the aortic valve prosthesis device, and computing a device depth of the aortic valve prosthesis device relative to an anatomical landmark.

Optionally, further comprising generating an overlay over the at least one fluoroscopic image indicating a first line indicating the anatomical landmark, a second line indicating the aortic valve prosthesis device, wherein the device depth is computed as distance between the first line and the second line.

Optionally, presenting comprises generating a graphical user interface (GUI) indicating a target range suitable for deployment of the aortic valve prosthesis device, and regions external to the target range indicating intensity of closeness to the target range, and a visual indication of the device depth within the target range or the regions external to the target range.

Optionally, analyzing comprises feeding the at least one fluoroscopic image into a machine learning model trained for segmenting the aortic valve prosthesis device within the at least one fluoroscopic image.

Optionally, the machine learning model is trained on a training dataset of a plurality of sample fluoroscopic images depicting sample aortic valve prosthesis devices, labelled with a ground truth segmentation of the sample aortic valve prosthesis device.

Optionally, further comprising monitoring for deviation of the device depth from a target range.

Optionally, the anatomical landmark comprises an annulus line of the aortic valve.

Optionally, the device depth is computed from a distal end location of the aortic valve prosthesis device to the anatomical landmark.

Optionally, further comprising determining at least one of a maximal distance and minimal distance of the distal end location of the aortic prosthesis device relative to the anatomical landmark based on historical values

Optionally, the device depth is computed from a proximal end location of the aortic valve prosthesis device to the anatomical landmark.

Optionally, the anatomical landmark comprises at least one of: coronary orifices and annulus line of the aortic valve.

Optionally, further comprising presenting the device depth on a display.

According to some embodiments of the present invention, there is provided an apparatus for guiding a trans-catheter aortic valve replacement intervention in a subject, comprising: at least one processor executing a code for: accessing at least one fluoroscopic image depicting an aortic valve prosthesis device in an aorta of the subject, the at least one fluoroscopic image excludes injected contrast, analyzing the at least one fluoroscopic image to identify a distal end of the aortic valve prosthesis device, and computing a device depth of the aortic valve prosthesis device relative to an anatomical landmark.

Optionally, further comprising code for generating an overlay over the at least one fluoroscopic image indicating a first line indicating the anatomical landmark, a second line indicating the aortic valve prosthesis device, wherein the device depth is computed as distance between the first line and the second line.

Optionally, presenting comprises generating a graphical user interface (GUI) indicating a target range suitable for deployment of the aortic valve prosthesis device, and regions external to the target range indicating intensity of closeness to the target range, and a visual indication of the device depth within the target range or the regions external to the target range.

Optionally, analyzing comprises feeding the at least one fluoroscopic image into a machine learning model trained for segmenting the aortic valve prosthesis device within the at least one fluoroscopic image.

Optionally, the machine learning model is trained on a training dataset of a plurality of sample fluoroscopic images depicting sample aortic valve prosthesis devices, labelled with a ground truth segmentation of the sample aortic valve prosthesis device.

Optionally, further comprising code for monitoring for deviation of the device depth from a target range.

Optionally, the anatomical landmark comprises an annulus line of the aortic valve.

Optionally, the device depth is computed from a distal end location of the aortic valve prosthesis device to the anatomical landmark.

Optionally, further comprising code for determining at least one of a maximal distance and minimal distance of the distal end location of the aortic prosthesis device relative to the anatomical landmark based on historical values

Optionally, the device depth is computed from a proximal end location of the aortic valve prosthesis device to the anatomical landmark.

Optionally, the anatomical landmark comprises at least one of: coronary orifices and annulus line of the aortic valve.

Optionally, further comprising code for presenting the device depth on a display.

According to some embodiments of the present invention, there is provided a computer implemented method of guiding a trans-catheter aortic valve replacement intervention in a subject, comprising: accessing at least one fluoroscopic image depicting an aortic valve prosthesis device in an aorta of the subject, analyzing the at least one fluoroscopic image for determining the aortic valve prosthesis device and an anatomical landmark, and computing of a tilt of the aortic valve prosthesis device relative to the anatomical landmark.

Optionally, further comprising generating an overlay over the at least one fluoroscopic image indicating a first line indicating the anatomical landmark, a second line indicating the aortic valve prosthesis device, wherein the tilt is computed as an angle between the first line and the second line.

Optionally, presenting comprises generating a graphical user interface (GUI) indicating a target range suitable for deployment of the aortic valve prosthesis device, and regions external to the target range indicating intensity of closeness to the target range, and a visual indication of the tilt within the target range or the regions external to the target range.

Optionally, analyzing comprises feeding the at least one fluoroscopic image into a machine learning model trained for segmenting the aortic valve prosthesis device within the at least one fluoroscopic image.

Optionally, the machine learning model is trained on a training dataset of a plurality of sample fluoroscopic images depicting sample aortic valve prosthesis devices, labelled with a ground truth segmentation of the sample aortic valve prosthesis device.

Optionally, the anatomical landmark comprises an annulus line of the aortic valve.

Optionally, further comprising presenting the tilt on a display.

According to some embodiments of the present invention, there is provided an apparatus for guiding a trans-catheter aortic valve replacement intervention in a subject, comprising: at least one processor executing a code for: accessing at least one fluoroscopic image depicting an aortic valve prosthesis device in an aorta of the subject, analyzing the at least one fluoroscopic image for determining the aortic valve prosthesis device and an anatomical landmark, and computing of a tilt of the aortic valve prosthesis device relative to the anatomical landmark.

Optionally, further comprising code for generating an overlay over the at least one fluoroscopic image indicating a first line indicating the anatomical landmark, a second line indicating the aortic valve prosthesis device, wherein the tilt is computed as an angle between the first line and the second line.

Optionally, presenting comprises generating a graphical user interface (GUI) indicating a target range suitable for deployment of the aortic valve prosthesis device, and regions external to the target range indicating intensity of closeness to the target range, and a visual indication of the tilt within the target range or the regions external to the target range.

Optionally, analyzing comprises feeding the at least one fluoroscopic image into a machine learning model trained for segmenting the aortic valve prosthesis device within the at least one fluoroscopic image.

Optionally, the machine learning model is trained on a training dataset of a plurality of sample fluoroscopic images depicting sample aortic valve prosthesis devices, labelled with a ground truth segmentation of the sample aortic valve prosthesis device.

Optionally, the anatomical landmark comprises an annulus line of the aortic valve.

Optionally, further comprising code for presenting the tilt on a display.

According to some embodiments of the present invention, there is provided a computer implemented method of guiding a trans-catheter aortic valve replacement intervention in a subject, comprising: accessing at least one fluoroscopic image depicting an aortic valve prosthesis device in an aorta of the subject, analyzing the at least one fluoroscopic image for determining the aortic valve prosthesis device and an anatomical landmark, and computing a depth and a tilt of the aortic valve prosthesis device relative to the anatomical landmark.

Optionally further comprising computing a width of the aortic valve prosthesis device.

According to some embodiments of the present invention, there is provided an apparatus for guiding a trans-catheter aortic valve replacement intervention in a subject, comprising: at least one processor executing a code for: accessing at least one fluoroscopic image depicting an aortic valve prosthesis device in an aorta of the subject, analyzing the at least one fluoroscopic image for determining the aortic valve prosthesis device and an anatomical landmark, and computing a depth and a tilt of the aortic valve prosthesis device relative to the anatomical landmark.

Optionally, further comprising code for computing a width of the aortic valve prosthesis device.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system for computing guidewire curvature and/or depth, angle, and/or tilt of an aortic valve prosthesis device, in accordance with some embodiments of the present invention;

FIG. 2 is a flowchart of a method for generating an image for guiding a trans-catheter aortic valve replacement intervention in a subject, in accordance with some embodiments of the present invention;

FIG. 3 is a flowchart of a method for training a machine learning model for generating an image for guiding a trans-catheter aortic valve replacement intervention in a subject, in accordance with some embodiments of the present invention;

FIG. 4 is an image that includes a marking indicating a detected valve annulus, and metrics computed based on the detected valve annulus, in accordance with some embodiments of the present invention;

FIG. 5 is another image that includes a marking indicating a detected valve annulus, and metrics computed based on the detected valve annulus, in accordance with some embodiments of the present invention;

FIG. 6 is an image that includes a detected lower plane of the subject aortic valve's cusps and a lower plane of the prosthetic device for computing a device depth, in accordance with some embodiments of the present invention;

FIG. 7 is a flowchart of a method of tracking an anatomical structure of a subject, in accordance with some embodiments of the present invention;

FIG. 8 is an image depicting an annulus line, a fiducial object, and a spatial relationship, in accordance with some embodiments of the present invention;

FIG. 9 is a flowchart of another exemplary method for tracking an anatomical structure of a subject, in accordance with some embodiments of the present invention;

FIG. 10 is a flowchart of a method of detecting an annulus on a non-contrast image by correlating with a 3D image, in accordance with some embodiments of the present invention;

FIG. 11 is a flowchart of a process for computing a distance between an annulus line and a distal end of a THV, in accordance with some embodiments of the present invention;

FIG. 12 is a flowchart of a method of computing a local curvature of a guidewire, in accordance with some embodiments of the present invention;

FIG. 13 is a schematic of an exemplary GUI, presenting an indication of a local curvature of a guidewire, in accordance with some embodiments of the present invention;

FIG. 14 is a flowchart of a method of computing width, depth, and/or tilt of an aortic valve prosthesis device, in accordance with some embodiments of the present invention;

FIG. 15 is a schematic of an exemplary GUI depicting an overlay over an aortic valve prosthesis device depicted in a fluoroscopic image, in accordance with some embodiments of the present invention;

FIG. 16 is a schematic of an exemplary GUI of a dashboard indicating device depth and/or tilt, in accordance with some embodiments of the present invention; and

FIGS. 17A-C depict an exemplary GUI (as described with reference to FIG. 15) that includes an overlay and a dashboard (as described with reference to FIG. 16) during various stages of a procedure for implanting an aortic valve prosthesis device, in accordance with some embodiments of the present invention.

### DETAILED DESCRIPTION

The present invention, in some embodiments thereof, relates to aortic valve replacement and, more specifically, but not exclusively, to systems and methods for guiding aortic valve replacement.

As used herein, the term annulus, which is detected based on images as described herein, may refer to a virtual annulus. The real annulus (of the Aortic valve) is a fibrous ring at the aortic orifice to the front and right of the atrioventricular aortic valve and is considered the transition point between the left ventricle and aortic root. The annulus is part of the fibrous skeleton of the heart. The annulus line and/or virtual annulus detected in images as described herein, refers to the location on the image expected to correspond to the location of the real annulus. The term annulus, lower annulus, annulus contour, contour of the annulus, annulus line, annulus plane, annulus of the heart, annulus of the valve (e.g., aortic valve, tricuspid valve, pulmonary valve) and virtual annulus may be used interchangeably. Alternatively, the contour of the annulus may refer to one or more curves of the annulus, such as a border between the contrast enhanced aortic root and surrounding tissues (without contrast). The annulus line may refer to a line drawn at nadir of cusps of the aortic valve. The annulus line may be computed using a 2D image. The annulus plane may be computed using a 3D image.

It is noted that the terms first anatomical structure and second anatomical structure are used interchangeably, and are not limiting. Rather, what is significant is whether a certain anatomical structure is visible on contrast enhanced images (and not visible or difficult to visualize on non-contrast images), or visible on non-contrast images (and not visible or difficult to visualize on contrast enhanced images). For example, calcification deposits are visible on non-contrast images but are difficult to visualize on contrast enhanced images. Sometimes calcification deposits are referred to as first anatomical structures, and sometimes as second anatomical structures, for example, according to the context of the description and/or in the summary.

As used herein, the term machine learning (ML) model is an exemplary implementation for processing images and/or features extracted from images, for example, a detector, one or more neural networks of various architectures (e.g., convolutional, fully connected, deep, encoder-decoder, recurrent, graph, combination of multiple architectures), support vector machines (SVM), logistic regression, k-nearest neighbor, decision trees, boosting, random forest, a regressor and the like. In some embodiments, other image processing approaches may be used in addition to, and/or alternatively to, the ML model, such as edge detection, feature extraction, masking, and the like.

An aspect of some embodiments of the present invention relate to systems, methods, computing devices, and/or code instructions (stored on a data storage device and executable by one or more processors) for computing curvature of a guidewire, optionally a stiff guidewire, depicted in a medical image. The medical image may be, for example, a 2D fluoroscopic image captured during a transcatheter procedure in which the guidewire is located within a lumen and/or organ of the body, for example, within the aorta and/or left ventricle such as during deployment of an aortic valve prosthesis device. The fluoroscopic image may be analyzed to identify the guidewire with local curvature, for example, by segmenting the guidewire from the image. A local curvature of the guidewire depicted in the fluoroscopic image is computed, optionally using the segmented guidewire. The segmentation may be computed by fitting a circle to the local curvature of the guidewire, and obtaining the radius of the fitted circle. An indication of the local curvature may be presented on a display, for example, as a coded overlay over the region of the guidewire presented within the image. Regions of the guidewire having curvature above a first threshold and/or radius below a second threshold representing risk of damage to tissue from high pressure exerted by the curvature may be indicated on the overlay, for example, coded by color and/or pattern and/or other visual indication.

The detected curvature may be indicative of a potential high pressure applied by the guidewire to the interior of the heart, which may adversely impact the conduction system of the heart. The detected curvature may be below the native aortic annulus plane, optionally within a chamber of the heart. By detecting the curvature indicating high pressure, the operator may reduce the curvature for reducing the pressure, for reducing likelihood of adversely impacting the conduction system of the heart.

As used herein, the term tilt and angle or orientation are used interchangeably in the context of the aortic valve prosthesis device relative to reference such as an anatomical landmark such as the annulus line, and/or relative to an external coordinate reference system.

An aspect of some embodiments of the present invention relate to systems, methods, computing devices, and/or code instructions (stored on a data storage device and executable by one or more processors) for computing a width and/or device depth and/or tilt of an aortic valve prosthesis device. A fluoroscopic image depicting the aortic valve prosthesis device in the aorta of the subject, optionally in proximity to a native aortic valve (or a previously deployed aortic valve prosthesis device), is obtained. The fluoroscopic image may exclude injected contrast. The fluoroscopic image may be analyzed to identify the aortic valve prosthesis device in the aorta. For example, the aortic valve prosthesis device is segmented, and/or detected by drawing a bounding box around the aortic valve prosthesis device. Optionally, two end tips of the aortic valve prosthesis device are identified, as points of the aortic valve prosthesis device that are most distal and most to the left and most to the right. A distal line may be drawn between the identified two end tips. The width may be measured as the length of the distal line. The device depth may be measured as the distance between the distal line and an annulus line representing the location of the annulus of the native aortic valve. When the distal line and the annulus line are non-parallel, the device depth may be an average of the distance between the distal line and the annulus line, for example, an average of the distance from each of the two end tips to the annulus line. The tilt may be measured as an angle between the distal line and the annulus line. The width and/or tilt and/or device depth may be presented on a display, optionally within a GUI, for example, within a dashboard having different coded regions such as a target region indication proper value for deployment, and/or hazard region indicating an improper value and deployment should not occur. At least some embodiments described herein address the technical problem of improving the safety of a procedure of deployment of an aortic valve prosthesis device, in a native aortic valve and/or within a previously implanted an aortic valve prosthesis device. At least some embodiments described herein address the technology of deployment of an aortic valve prosthesis device.

While Transcatheter Aortic Valve Implantation (TAVI) of an aortic valve prosthesis device is generally considered a safe and effective procedure, there can be certain challenges or complications associated with its performance. Here are some of the potential problems that can arise during or after a TAVI procedure, which at least some embodiments described herein may reduce or prevent by proper deployment at, and/under guidance of, target width and/or device depth and/or tilt and/or curvature:
Valve-related complications: Complications related to the aortic valve prosthesis device itself. These may include valve mal-positioning, valve migration, valve dislodgement, valve embolization (detachment of the valve), valve leakages, or valve dysfunction. At least some embodiments described herein may improve deployment of the aortic valve prosthesis device, which may reduce these complications.
Conduction disturbances: During a TAVI procedure, the electrical conduction system of the heart may be damaged, such as by a high curvature of the guidewire, leading to conduction abnormalities such as heart block or arrhythmias. This may require further intervention, such as implantation of a pacemaker or other devices, to maintain normal heart rhythm. Embodiments described herein may monitor curvature of the guidewire, enabling reducing and/or preventing high curvature of the guidewire.
Paravalvular leak: In some cases, a small amount of blood may leak around the aortic valve prosthesis device, which is known as paravalvular leak. While mild leakage may not cause significant symptoms or require intervention, moderate to severe leakage can affect the overall effectiveness of the procedure and may necessitate further treatment or valve replacement. Using the device depth and/or width and/or tilt described herein, the operator may improve deployment of the aortic valve prosthesis device for reducing or preventing paravalvular leaks.
Correct 3D positioning: Accurate positioning of the aortic valve prosthesis device is critical for the success of a TAVI procedure. If the aortic valve prosthesis device is mal-positioned, it can result in inadequate sealing, paravalvular leaks, or impaired function.
Pressure gradients and turbulent flow: The aim of TAVI procedure is to restore normal blood flow across the aortic valve and reduce pressure gradients. However, in some cases, residual pressure gradients may persist, limiting the effectiveness of the procedure. Additionally, the implantation of an artificial valve can disrupt the normal flow patterns within the aorta, leading to turbulent flow. While mild turbulence is generally well-tolerated, significant turbulence can increase the risk of complications such as blood clots or hemolysis. Using the device depth and/or width and/or tilt described herein, the operator may improve deployment of the aortic valve prosthesis device for reducing or preventing improper pressure gradients and turbulent flow.
Thrombosis: Thrombosis refers to the formation of blood clots on or around the artificial valve. This can occur due to factors such as inadequate anticoagulation therapy, improper valve sizing or positioning, or underlying patient conditions. Clots can obstruct the valve, impede blood flow, and increase the risk of stroke or other cardiovascular events. Using the device depth and/or width and/or tilt described herein, the operator may improve deployment of the aortic valve prosthesis device for reducing or preventing thrombosis.
Valve mechanical failures: These failures may include structural valve deterioration, valve leaflet dysfunction, or fracture of the valve frame. Such failures can lead to valve dysfunction, impaired blood flow, or other complications. Close monitoring and regular follow-up appointments are important to detect any signs of valve mechanical failures early on. Using the device depth and/or width and/or tilt described herein, the operator may improve deployment of the aortic valve prosthesis device for reducing or preventing mechanical failure.

At least some embodiments described herein provide a solution to the aforementioned technical problem, and/or improve the aforementioned technical field, by dynamically computing curvature of a guidewire (e.g., used to guide the aortic valve prosthesis device) and/or dynamically computing a width and/or tilt and/or device depth of the aortic valve prosthesis device using fluoroscopic images captured during the procedure. Optionally, a GUI optionally including an overlay and/or dashboard, is dynamically updated to indicate whether the curvature and/or width and/or tilt and/or device depth is within a target range suitable for deployment and/or for reduced risk to tissue damage (e.g., curvature) or external to the target range indicating less optimal values and/or a contra-indication to deployment.

At least some embodiments described herein address the technical problem of guiding a trans-catheter aortic valve replacement procedure. Such procedures are commonly performed under fluoroscopic imaging, which makes it difficult to visualize features of the prosthetic valve that is being deployed and/or anatomical features of the aorta and/or heart in which the prosthetic valve is being deployed. Administration of contrast enhances the visualization, but the amount of contrast that can be administered is physiologically limited, since too much contrast can harm the body of the subject. At least some embodiments described herein improve the medical technology of guiding a trans-catheter aortic valve replacement and/or improve the technology of machine learning.

While procedures for transcatheter implantation of an aortic valve procedure are substantially less invasive than open heart surgery, the lack of line-of-sight visualization of the prosthetic heart valve and the native valve presents challenges, because the physician cannot see the actual orientation of the prosthetic heart valve during the implantation procedure. Correct positioning of the prosthetic heart valve is achieved using radiographic imaging, which yields a two-dimensional image of the viewed area. The physician must interpret the image correctly in order to properly place the prosthetic heart valve in the desired position. Failure to properly position the prosthetic heart valve sometimes leads to migration of the prosthetic heart valve or to improper functioning. Proper placement of the prosthetic heart valve using radiographic imaging is thus critical to the success of the implantation.

An aspect of some embodiments of the present invention relates to systems, methods, computing devices, and/or code instructions for automatically detecting an annulus within a native valve (e.g., aortic valve) for computing a device depth and/or tilt of an aortic valve prosthesis device relative to the annulus. The annulus may be detected on a contrast image that depicts injected contrast (e.g., into the aortic root). The annulus is difficult to directly visualize or cannot be directly visualized on images that exclude contrast, making it difficult to determine the device depth and/or tilt relative to the annulus.

At least some embodiments described herein address the technical problem of detecting the annulus of a native valve (e.g., aortic valve) on images that exclude contrast for computing a device depth and/or tilt of an aortic valve prosthesis device relative to the annulus. At least some embodiments described herein improve the technical field of image processing by detecting the annulus of the native valve (e.g., aortic valve) on images that exclude contrast for computing a device depth and/or tilt of an aortic valve prosthesis device relative to the annulus. Determining the location of the annulus is clinically significant, for example, for computing a device depth and/or tilt of an aortic valve prosthesis device relative to the annulus for deployment thereof. Using standard approaches, the location of the annulus may be estimated by repeated contrast injections. It is clinically desirable to reduce injection of contrast into the blood of a subject, since for example, contrast places extra stress on the kidneys, there is a maximum amount of contrast that can be injected, and excessive contrast may be associated with other risks such as allergic reaction.

At least some embodiments described herein provide a technical solution to the aforementioned technical problem, and/or improve the aforementioned technical field, and/or improve upon the aforementioned prior approaches, by enabling detecting and/or tracking of the annulus on images that exclude contrast for computing a device depth and/or tilt of an aortic valve prosthesis device relative to the annulus.

An aspect of some embodiments of the present invention relates to systems, methods, computing devices, and/or code instructions, for detecting an annulus line on non-contrast images for computing a device depth and/or tilt of an aortic valve prosthesis device relative to the annulus, for example, 2D fluoroscopic images of a native aortic valve.

An aspect of some embodiments of the present invention relates to systems, methods, computing devices, and/or code instructions for computing a device depth of a THV from one or more images, optionally 2D fluoroscopic images. The image depicts a partially expanded THV positioned within the native aortic valve. The device depth is computed as a distance between an annulus line (also referred to herein as lower annulus) and a distal end of the THV. The device depth is used to position the THV.

At least some embodiments described herein address the technical problem of computing a device depth of a THV from images, such as 2D fluoroscopic images (contrast and/or non-contrast). The device depth is used to position the THV. In particular, the problem is to determine the device depth when the image depicts a partially expanded THV. At least some embodiments described herein improve the technical field of image processing by automatically computing a device depth of a THV from images.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random-access memory (RAM), a read-only memory (ROM), an erasable programmable read-only memory (EPROM or Flash memory), a static random-access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

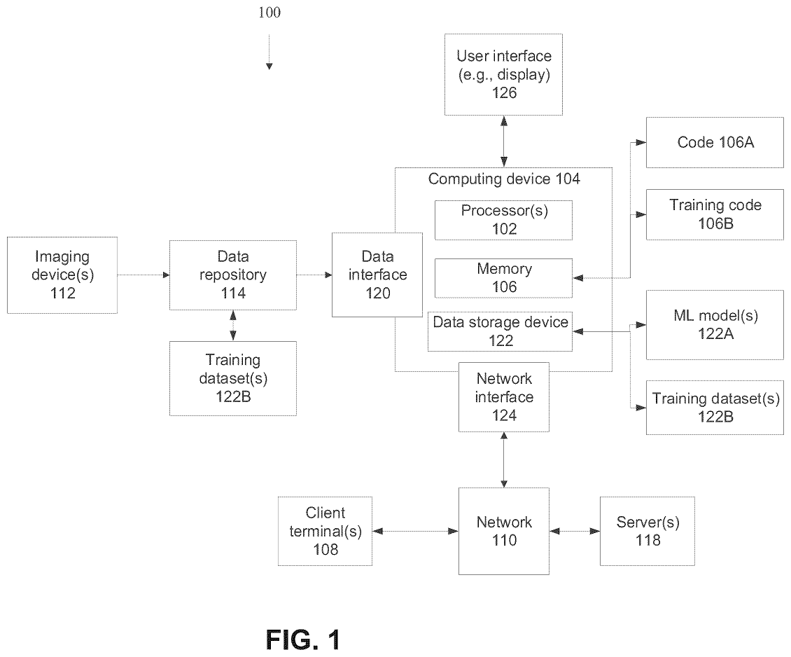

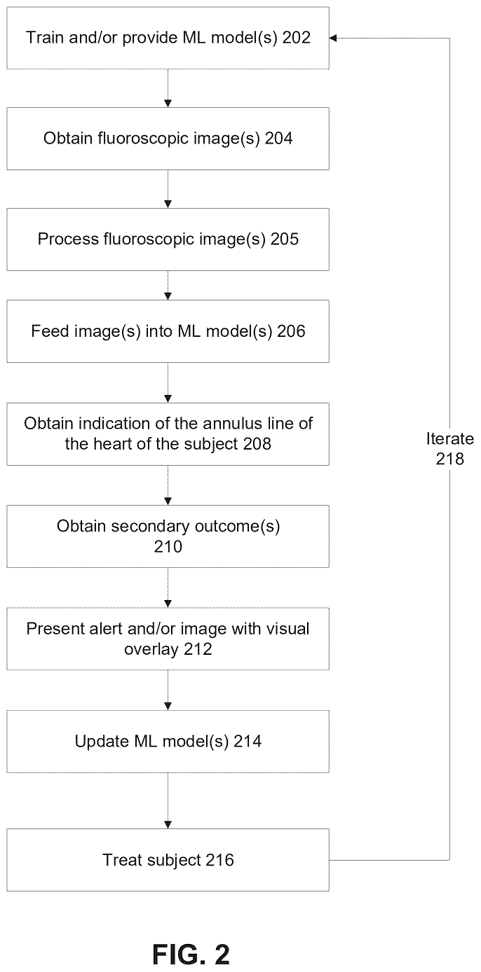

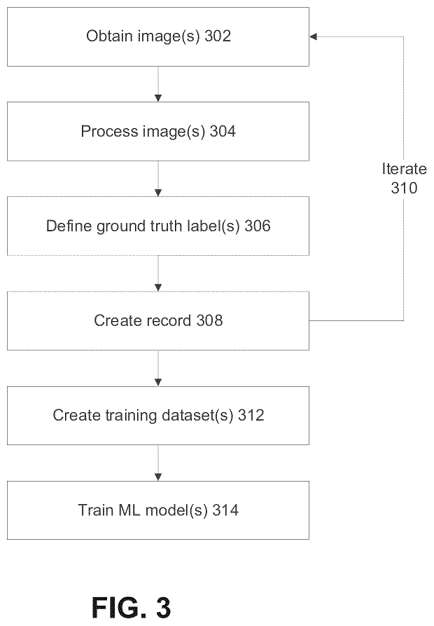

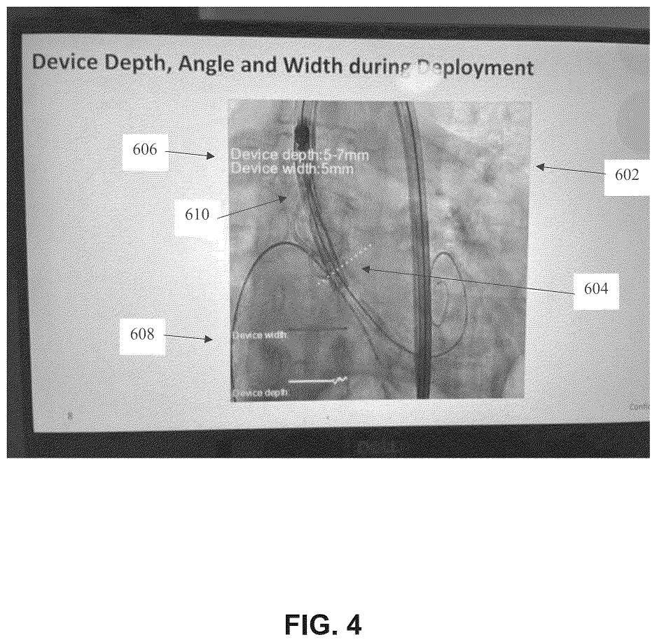

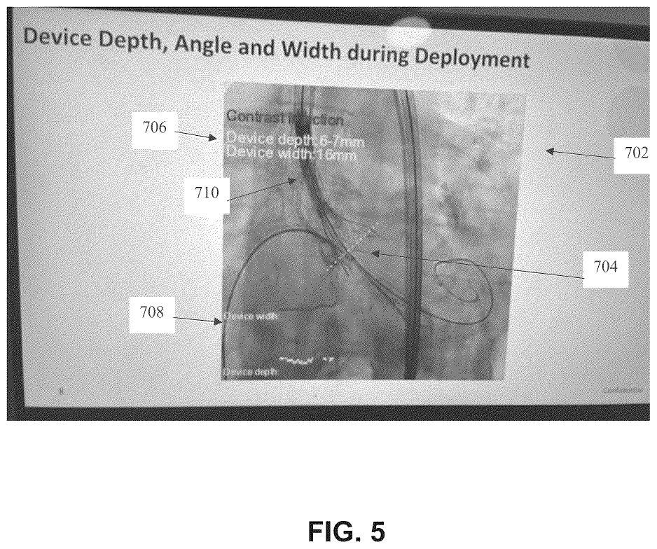

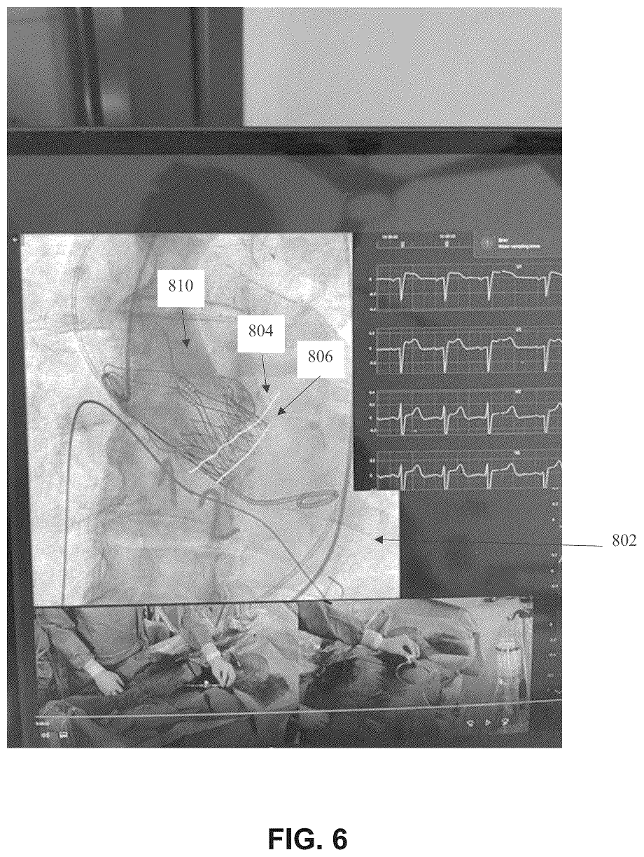

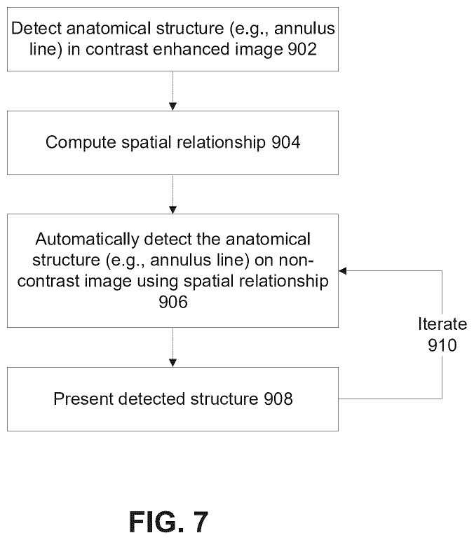

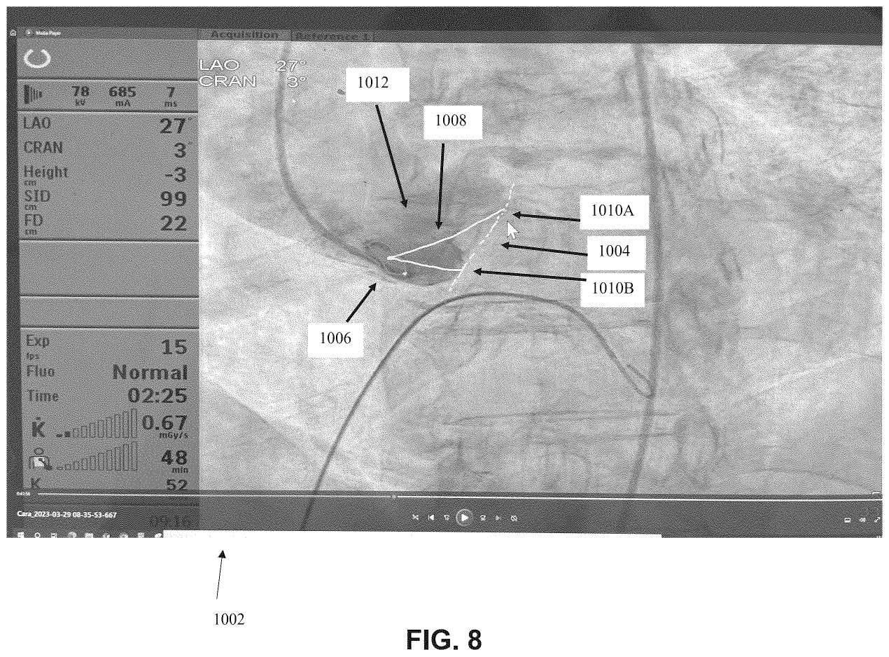

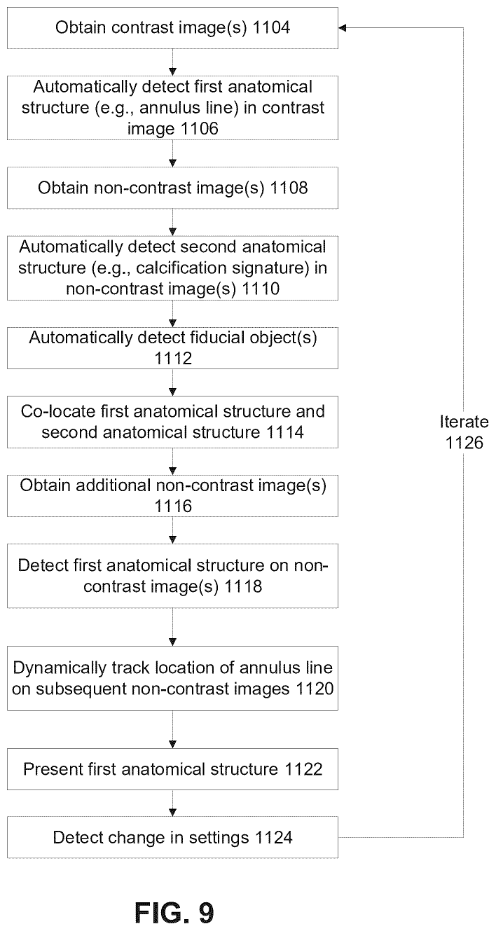

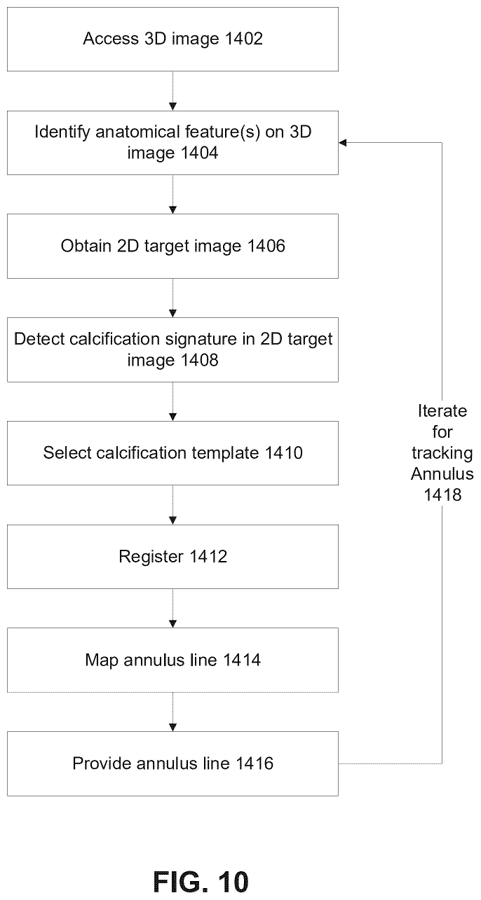

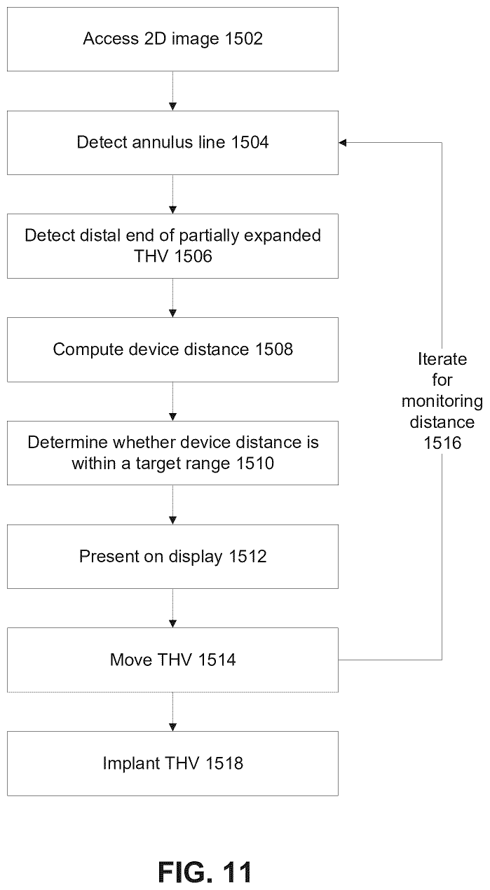

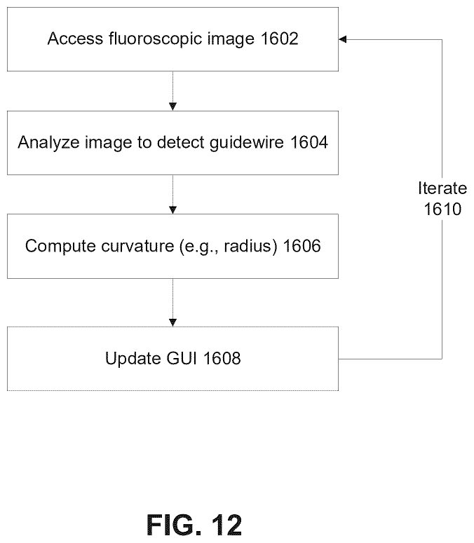

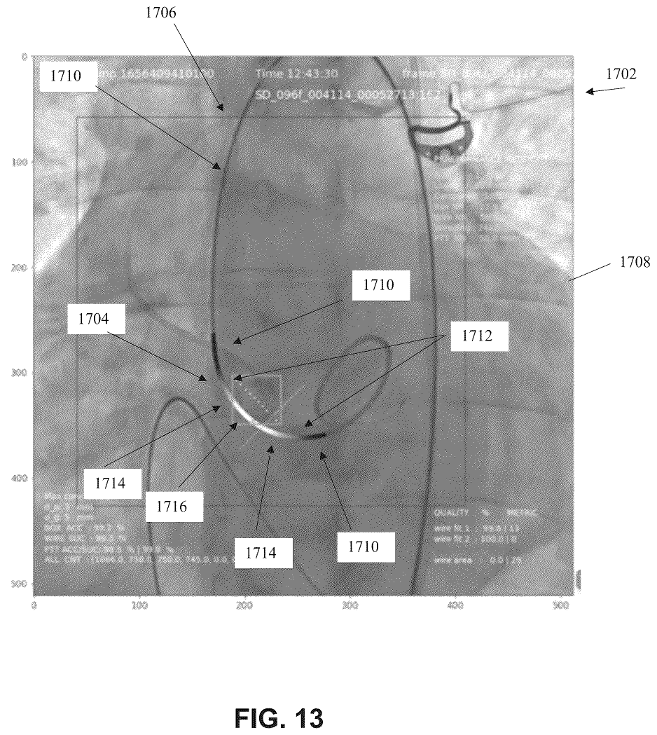

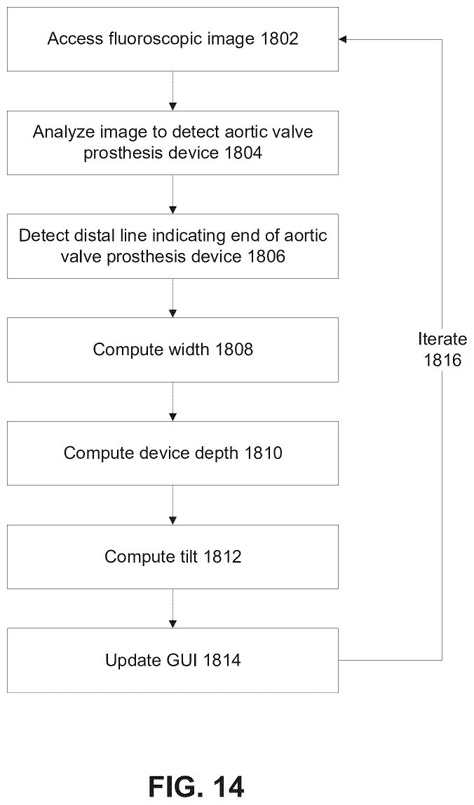

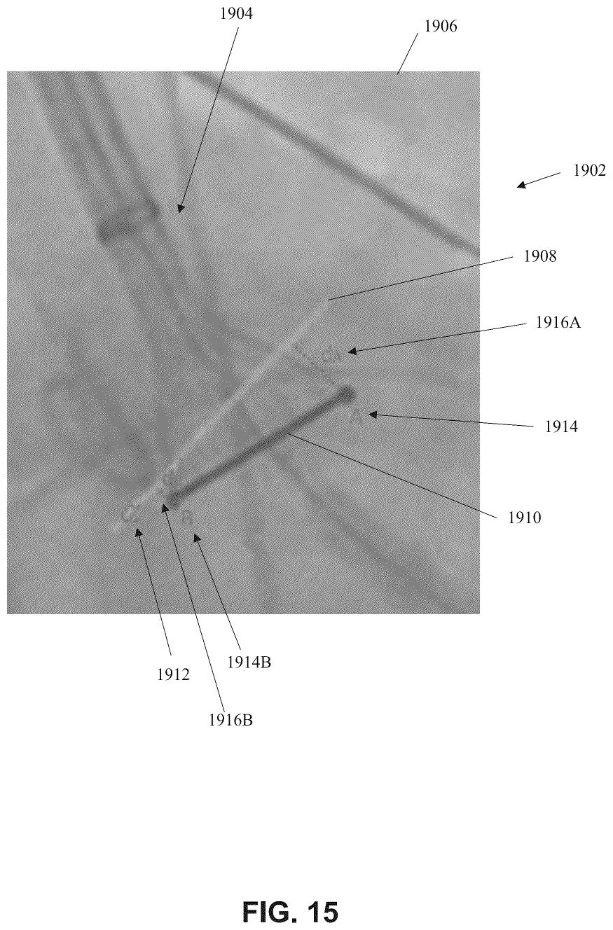

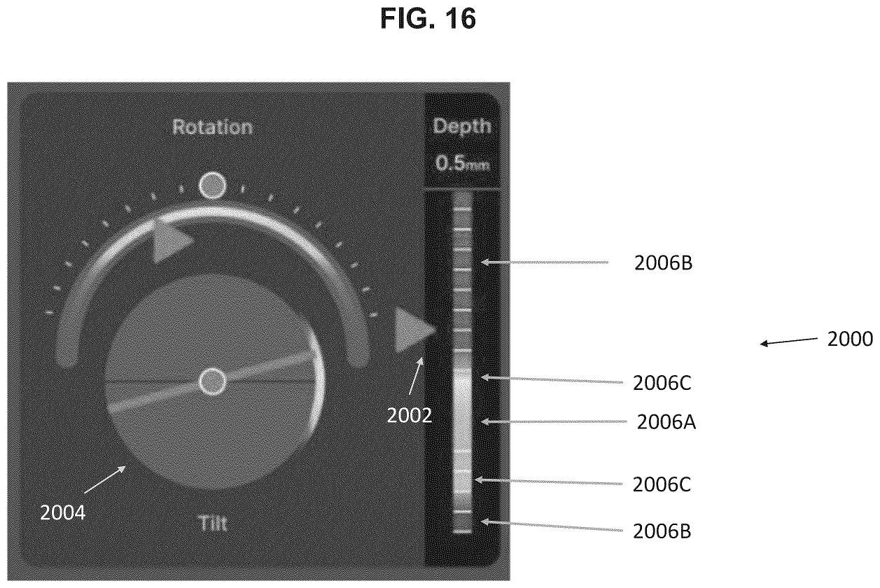

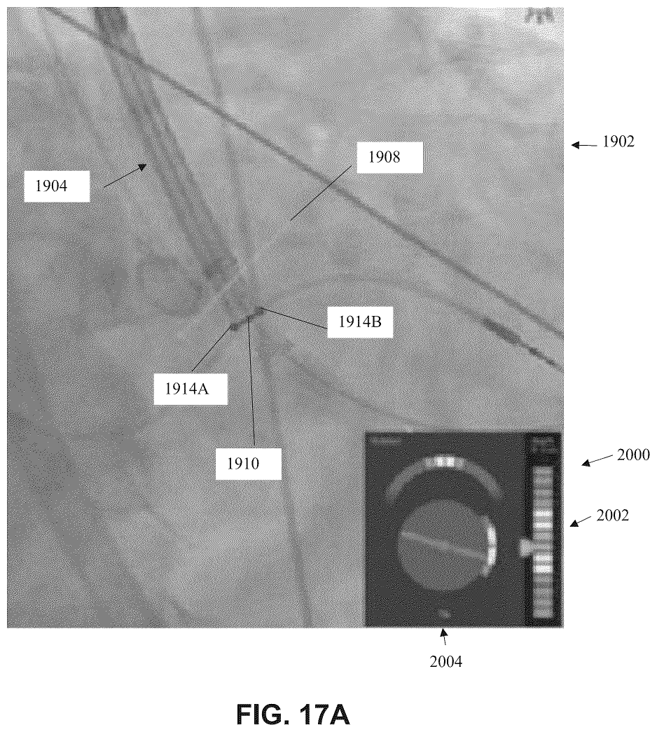

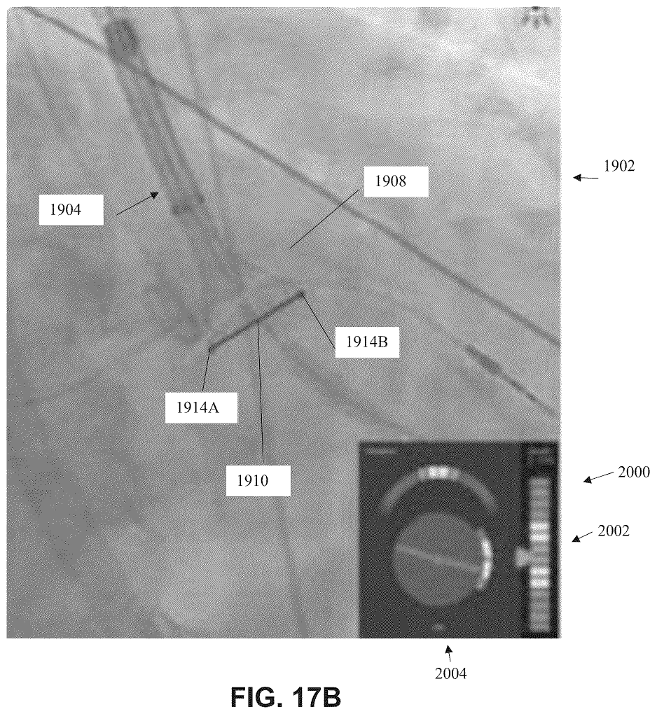

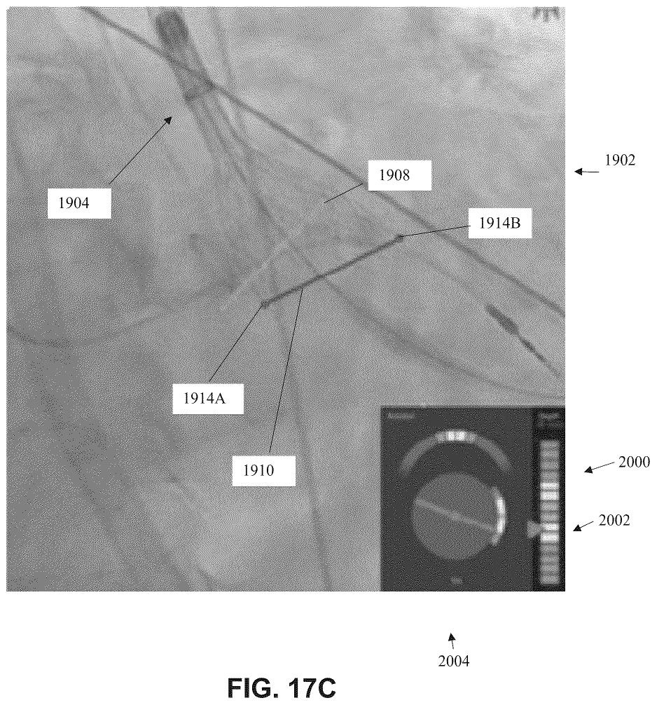

Reference is now made to FIG. 1, which is a block diagram of components of a system 100 for computing guidewire curvature and/or depth, angle, and/or tilt of an aortic valve prosthesis device, in accordance with some embodiments of the present invention. Reference is also made to FIG. 2, which is a flowchart of a method for generating an image for guiding a trans-catheter aortic valve replacement intervention in a subject, in accordance with some embodiments of the present invention. Reference is also made to FIG. 3, which is a flowchart of a method for training a machine learning model for generating an image for guiding a trans-catheter aortic valve replacement intervention in a subject, in accordance with some embodiments of the present invention. Reference is also made to FIG. 4, which is an image 602 that includes a marking 604 indicating a detected valve annulus, and metrics 606 608 computed based on the detected valve annulus, in accordance with some embodiments of the present invention. Reference is also made to FIG. 5, which is another image 702 that includes a marking 704 indicating a detected valve annulus, and metrics 706 708 computed based on the detected valve annulus, in accordance with some embodiments of the present invention. Reference is also made to FIG. 6, which is an image 802 that includes a detected lower plane of the subject aortic valve's cusps 804 and a lower plane of the prosthetic device 806 for computing a device depth, in accordance with some embodiments of the present invention. Reference is also made to FIG. 7, which is a flowchart of a method of tracking an anatomical structure of a subject, in accordance with some embodiments of the present invention. Reference is also made to FIG. 8, which is an image 1002 (e.g., fluoroscopic images) depicting an annulus line 1004, a fiducial object 1006, and a spatial relationship 1008, in accordance with some embodiments of the present invention. Reference is also made to FIG. 9, which is a flowchart of another exemplary method for tracking an anatomical structure of a subject, in accordance with some embodiments of the present invention. Reference is also made to FIG. 10, which is a flowchart of a method of detecting an annulus on a non-contrast image by correlating with a 3D image, in accordance with some embodiments of the present invention. Reference is also made to FIG. 11, which is a flowchart of a process for computing a distance between an annulus line and a distal end of a THV, in accordance with some embodiments of the present invention. Reference is also made to FIG. 12, which is a flowchart of a method of computing a local curvature of a guidewire, in accordance with some embodiments of the present invention. Reference is also made to FIG. 13, which is a schematic of an exemplary GUI 1702, presenting an indication of a local curvature 1704 of a guidewire 1706, in accordance with some embodiments of the present invention. Reference is also made to FIG. 14, which is a flowchart of a method of computing width, depth, and/or tilt of an aortic valve prosthesis device, in accordance with some embodiments of the present invention. The width, depth, and/or tilt may information to assist a user with optimal implantation of the aortic valve prosthesis device, for example, with reference to an aortic annulus line. Reference is also made to FIG. 15, which is a schematic of an exemplary GUI 1902 depicting an overlay over an aortic valve prosthesis device 1904 depicted in a fluoroscopic image 1906, in accordance with some embodiments of the present invention. Reference is also made to FIG. 16, which is a schematic of an exemplary GUI of a dashboard 2000 indicating device depth 2002 and/or tilt 2004, in accordance with some embodiments of the present invention. Reference is also made to FIGS. 17A-C, which depict an exemplary GUI 1902 (as described with reference to FIG. 15) that includes an overlay and a dashboard 2000 (as described with reference to FIG. 16) during various stages of a procedure for implanting an aortic valve prosthesis device, in accordance with some embodiments of the present invention.

System 100 may implement the acts of the method described with reference to FIGS. 2-17C, optionally by a hardware processor(s) 102 of a computing device 104 executing code instructions stored in a memory 106.

Computing device 104 may be implemented as, for example, a client terminal, a server, a virtual server, a radiology workstation, an angiography workstation, a virtual machine, a computing cloud, a mobile device, a desktop computer, a thin client, a Smartphone, a Tablet computer, a laptop computer, a wearable computer, glasses computer, and a watch computer. Computing 104 may include an advanced visualization process that sometimes is add-on to an angiography workstation and/or other devices for presenting overlays on fluoroscopic images for guiding a trans-catheter aortic valve replacement intervention in a subject.

Computing device 104 may include locally stored software that performs one or more of the acts described with reference to FIGS. 2-17C, and/or may act as one or more servers (e.g., network server, web server, a computing cloud, virtual server) that provides services (e.g., one or more of the acts described with reference to FIGS. 2-17C) to one or more client terminals 108 (e.g., remotely located angiography workstations, remote picture archiving and communication system (PACS) server, remote electronic medical record (EMR) server) over a network 110, for example, providing software as a service (Saas) to the client terminal(s) 108, providing an application for local download to the client terminal(s) 108, as an add-on to a web browser and/or a medical imaging viewer application, and/or providing functions using a remote access session to the client terminals 108, such as through a web browser.

As used herein, the terms imaging device (e.g., 112) and image sensor are used interchangeably.

Different architectures based on system 100 may be implemented. In one example, computing device 104 provides centralized services. Training of one or more ML models 122A may be performed centrally by computing device 104, as described herein. Computing device 104 may train generic and/or customized ML models. Inference of locally captured fluoroscopic images (e.g., captured by imaging device 112) for computing guidewire curvature and/or depth, angle, and/or tilt of an aortic valve prosthesis device may be centrally performed by computing device 104. Alternatively, training is performed by another computing device, and inference is centrally performed by computing device 104. Fluoroscopic images may be provided to computing device 104 for centralized inference by the trained ML model(s) 122A. Images may be provided to computing device 104, for example, via an API, a local application, and/or transmitted using a suitable transmission protocol. Alternatively, analysis is done by non-ML approaches, such as image processing. The outcome of the inference and/or image processing, such as computing guidewire curvature and/or depth, angle, and/or tilt of an aortic valve prosthesis device may be provided, for example, to client terminal(s) 108 for presentation on a display and/or local storage, stored in an electronic medical record (e.g., hosted by server 118), and/or stored by computing device 104. In another example, computing device 104 provides centralized training of the ML model(s) 122A, using records to create one or more training datasets 122B provided by different client terminals 108 and/or servers 118. For example, training datasets originating from different hospitals, and/or training dataset for different imaging modalities, and/or for different types of prosthetic valves and/or for different cases such as deployment within a native aortic valve or deployment of another valve within an existing deployed prosthetic valve and/or different types of guidewires. Respective generated ML models 122A may be provided to the corresponding remote devices (e.g., client terminal(s) 108 and/or server(s) 118) for local use. For example, each hospital uses the ML model created from their own training dataset for evaluation of new images captured at the respective hospital, and/or different ML models are locally used to evaluate medical images for different types of prosthetic valves.

Imaging device 112 provides the images, which may be included in training dataset(s) 122A and/or for inference. Optionally, imaging device 112 is a 2D fluoroscopic and/or x-ray machine, as commonly used in cardiac catheterization labs. Alternatively or additionally, imaging device 112 includes a 2D, 3D, and/or 4D imaging device, such as for capturing pre-procedure images as described herein, for example, a CT machine or a MRI machine. The 3D CT image may be evaluated, and/or 2D slices may be extracted from the 3D CT scan, for example, for training and/or for comparison with 2D real time acquired fluoroscopic images.

Training dataset(s) 122B may be stored in a data repository 114 and/or data storage device 122, for example, a storage server, a computing cloud, virtual memory, and a hard disk. Training dataset(s) 122B are used to train the ML model(s) 122A, as described herein. It is noted that training dataset(s) 122B may be stored by a server 118, accessibly by computing device 104 over network 110.

Where non-ML models approaches such as image processing approaches are used, the training datasets may be used for determining how to configure the image processing approaches.

Computing device 104 may receive images and/or records of the training dataset(s) 122B from imaging device 112 and/or data repository 114 using one or more data interfaces 120, for example, a wire connection (e.g., physical port), a wireless connection (e.g., antenna), a local bus, a port for connection of a data storage device, a network interface card, other physical interface implementations, and/or virtual interfaces (e.g., software interface, virtual private network (VPN) connection, application programming interface (API), software development kit (SDK)).

Hardware processor(s) 102 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include one or more processors (homogenous or heterogeneous), which may be arranged for parallel processing, as clusters and/or as one or more multi core processing units.

Memory 106 (also referred to herein as a program store, and/or data storage device) stores code instruction for execution by hardware processor(s) 102, for example, a random-access memory (RAM), read-only memory (ROM), and/or a storage device, for example, non-volatile memory, magnetic media, semiconductor memory devices, hard drive, removable storage, and optical media (e.g., DVD, CD-ROM). For example, memory 106 may store image processing code 106A that implement one or more acts and/or features of the method described with reference to FIGS. 2, and 4-17C and/or training code 106B that execute one or more acts of the method described with reference to FIG. 3.

Computing device 104 may include a data storage device 122 for storing data, for example, one or more trained ML models 122A as described herein and/or one or more training datasets 122B as described herein. Data storage device 122 may be implemented as, for example, a memory, a local hard-drive, a removable storage device, an optical disk, a storage device, and/or as a remote server and/or computing cloud (e.g., accessed over network 110). It is noted that 122A-B may be stored in data storage device 122, with executing portions loaded into memory 106 for execution by processor(s) 102.

Computing device 104 may include a network interface 124 for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations. Computing device 104 may access one or more remote servers 118 using network 110, for example, to obtain and/or provide training dataset(s) 116, an updated version of code 106A, training code 106B, and/or the trained ML model(s) 122A.

It is noted that data interface 120 and network interface 124 may exist as two independent interfaces (e.g., two network ports), as two virtual interfaces on a common physical interface (e.g., virtual networks on a common network port), and/or integrated into a single interface (e.g., network interface). Computing device 104 may communicate using network 110 (or another communication channel, such as through a direct link (e.g., cable, wireless) and/or indirect link (e.g., via an intermediary computing device such as a server, and/or via a storage device) with one or more of:
Client terminal(s) 108, for example, when computing device 104 acts as a server providing image analysis services (e.g., SaaS) to remote angiography terminals, for analyzing remotely obtained fluoroscopic images using the trained ML model(s) 122A. Training dataset(s) 122B may be created based on fluoroscopic images received from one or more client terminals 108.
Server 118, for example, implemented in association with a PACS, which may store image for training dataset(s) 122B and/or may store captured fluoroscopic images for inference. Training dataset(s) 122B may be created from fluoroscopic images stored by server 118.
Imaging device 112 and/or data repository 114 that store fluoroscopic images acquired by imaging device 112. The acquired fluoroscopic images may be fed into trained ML model(s) 122A for inference. Training dataset(s) 122B may be created based on fluoroscopic images obtained from one or more imaging devices 112.

Computing device 104 and/or client terminal(s) 108 and/or server(s) 118 include and/or are in communication with a user interface(s) 126 that includes a mechanism designed for a user to enter data (e.g., manually define ground truth) and/or view the indication outputted by the ML model(s) such as an overlay over the input image. Exemplary user interfaces 126 include, for example, one or more of, a touchscreen, a display, a keyboard, a mouse, and voice activated software using speakers and microphone.

Referring now back to FIG. 2, in some embodiments, the secondary outcome(s) described with reference to 210 are computed without computing the annulus line as described with reference to 208, for example, guidewire curvature, and/or width, and optionally tilt, and/or device depth of the aortic valve prosthesis device. In some embodiments, the user (e.g., the physician) may indicate the annulus line, e.g., by a user interface.

In other embodiments the secondary outcome(s) described with reference to 210 are computed relative to the annulus line as described with reference to 208, for example, tilt, and/or device depth of the aortic valve prosthesis device.

At 202, one or more machine learning models are trained and/or trained ML models are accessed. An exemplary approach for training the ML model is described for example with reference to FIG. 3.

In some embodiments, the ML model may be trained for generating an indication for a correct implantation for implanting the aortic valve prosthesis device in the aorta, in response to an input of fluoroscopic images without contrast. In some embodiments, the ML model may be trained for generating an indication of the annulus line of the heart of the subject, in response to an input of fluoroscopic images without contrast.

The correct implantation area (it has several relevant places that might be interesting to know about, in the valve area) for the valve is not visible unless contrast material is injected. The ML model may find places (on the dye-image) and continue marking them on the fluoroscopy image as the procedure progresses.

The ML model decides what landmarks it can use and what it cannot. Pre-operative CT can help with that. In some embodiments, the landmarks include one or more of: anatomical structures around the annulus line and signature of calcification presence in the heart.

The machine learning model may be trained on a training dataset of fluoroscopic image with injected contrast that depicts at least one of the aortic valve prostheses and the indication for the annulus line.

In some embodiments, the ML model may be fine-tuned on the actual signature of calcification of the specific patient obtained from pre-procedure CT image of the patient and/or fluoroscopic images taken when the patient was injected with contrast. In some embodiments, the ML model may learn the relation, e.g., spatial relationship, between the annulus line and the signature of calcification of the specific patient. During the procedure, the signature of calcification of the specific patient may be detected and/or identified from fluoroscopic images taken when no contrast was injected and the annulus line of the patient may be automatically detected based on the spatial relationship between the annulus line and the signature of calcification of the specific patient. At 204, the processor obtains one or more fluoroscopic images (also referred to herein as current image(s)) and/or target image, depicting an aortic valve prosthesis device in an aorta of the subject). The fluoroscopic images excluding injected contrast, which makes it difficult or impossible to sufficiently visualize the aortic valve prosthesis and/or the indication for determining a site for implanting the aortic valve prosthesis device and/or the annulus line.

At 205, the processor may pre-process the one or more fluoroscopic images.

Optionally, the processor processes the fluoroscopic image, by correcting its alignment, and feeding the corrected image into the ML model. The processor may process the at least one fluoroscopic image by comparing the fluoroscopic angulation of the at least one fluoroscopic image to at least one image of the training dataset used to train the ML model and/or at least one image acquired before the procedure that excludes the valve prosthesis. The processor may correct a definition of correct alignment use for the at least one fluoroscopic image and/or correct incorrect alignment of the at least one fluoroscopic image.

Alternatively or additionally, the processor extracts landmark features from the fluoroscopic images. The landmark features may be fed into the ML model. For example, the processor identifies multiple landmarks on the fluoroscopic image (e.g., by feeding into another ML model, and/or by image processing code) which are then fed into the ML model to obtain the indication of a native aortic valve annulus. For example, the indication is a detection of an annulus of a native aortic valve, and the landmarks include one or more of: signature of calcification presence in the heart, e.g., in the vicinity of the annulus line, location of commissures of the native aortic valve, entry to at least one coronary artery, and location of a pigtail catheter positioned in one of the coronary cusps of the native aortic valve. The signature of calcification presence in the heart, e.g., in the vicinity of the annulus line, may be calculated from CT or MRI image of the subject, taken pre-procedure. The signature of calcification presence in the heart, e.g., in the vicinity of the annulus line, may be calculated from fluoroscopic image of the subject, taken when contrast was injected. In some embodiment, the ML model may learn the annulus line position in the heart in relation to such calcification presence; as the calcification presence is detected on the image also when no contrast is injected.

Optionally, the processor extracts a location of calcification presence. The processor may feed the location into the ML model for generating an outcome indicating a target anatomical feature, such as location of the aortic plane or annulus line. In other words, the processor may automatically track a first anatomical structure (e.g., calcification presence) in images taken during the procedure, e.g., when no contrast was injected, and used such first anatomical structure for co-locating a second anatomical structure (e.g., annulus line) to present such second anatomical structure on a display (e.g., as an overlay on the images). The second anatomical structure may be displayed on the display continuously during the procedure.

Optionally, the processor extracts two or more landmarks in the image. The two or more landmarks may be fed into the ML model for obtaining the annulus line. Alternatively, the location of the two or more landmarks is not explicitly extracted as a feature, but serves as an implied feature when the two or more landmarks are depicted in the current image.

At 206, the processor may feed the fluoroscopic image into the machine leaning model. Alternatively, the processor feeds the fluoroscopic image into two processing paths. The processor may feed the fluoroscopic image into a first processing path that includes a first ML model that generates a first outcome of an anatomical landmark of the subject, and the processor feeds the same fluoroscopic image into a second processing path that includes a second ML model that generates a second outcome of a physical landmark of the aortic valve prosthesis. The two processing paths enable computing a distance metric as a difference between the first outcome and the second outcome. Optionally, the anatomical landmark of the first outcome is a location of an aortic plane, and the physical landmark of the second outcome is a distal end of the artificial valve prosthesis.

Alternatively, the processor feeds the fluoroscopic image into two processing paths. The processor may feed the fluoroscopic image into a first processing path that includes a first ML model that generates a first outcome of an anatomical landmark of the subject (e.g., calcification presence), and the processor feeds the same fluoroscopic image into a second processing path that includes a second ML model that generates a second outcome of the annulus line of the heart of the subject.

The first ML model may be trained on a first training dataset of records. Each record may include an image in which a region that includes an ascending aorta is masked and a first ground truth label of a location of the anatomical landmark. The second ML model is trained on a second training dataset of records. Each record includes a second complete image with ascending aorta and a second ground truth label of a location of the physical landmark.

At 208, the processor may obtain an indication of the annulus line of the heart of the subject. Optionally, the indication is for a correct implantation for implanting the aortic valve prosthesis device in the aorta, optionally for computing the device depth and/or tilt and/or width of an aortic valve prosthesis device. Optionally, the processor may obtain an indication of the annulus line of the heart of the subject from a user (e.g., physician), e.g., by user interface. Optionally, the processor may obtain (e.g., by computing) an indication of the annulus line of the heart of the subject as described herein, for example in reference to one or more of FIGS. 2, 4, 5, 6, 7, 8, 9, 10, and 11.

In some embodiments, feature 208 is not implemented, i.e., the annulus line is not obtained and/or computed.

Exemplary approaches for detecting the annulus line, optionally on non-contrast images, are described herein, for example, with reference to FIG. 7, FIG. 9, FIG. 10.

In some embodiments, an ML model is not used and the spatial relationship between the annulus line and the signature of calcification of the specific patient and/or the signature of calcification of the specific patient may be detected by image processing methods, for example: by cross correlation the image (taken when no contrast was injected) with template of calcium signature of the subject. Once the calcium signature of the subject was identified in the image (e.g., its position), the annulus line of the subject may be calculated based on the spatial relationship identified.

The indication may be an overlay, and/or the input image with overlay. The indication may be text, code, drawing, and the like for generating an alert and/or overlay.

The indication of the annulus line of the heart of the subject is an exemplary implementation. Other structures within a native valve of a subject may be indicated as well.

Optionally, the indication may be at least one anatomical landmark. For example, feature in an anatomical region of the aorta for implantation of the aortic valve prosthesis device. In another example, the anatomical landmark is a location of the entry to the two-coronary artery (e.g., for the purpose of relating the image to the pre procedure CT for indicating beyond commissural alignment of the orientation of the artificial valve with regard to the subject's coronary artery openings, a step termed coronary alignment).

Alternatively or additionally, the indication may be a depth of the artificial valve prosthesis relative to an anatomical landmark of the subject (also referred to herein as device depth). The anatomical landmark may be a lower plane of an annulus (cusps) of an aortic valve of the subject. The depth may be from the anatomical landmark to a lower plane of the artificial valve prosthesis. Optionally, the processor generates an alert indicating correct placement when the depth is between about 3-5 millimeters (mm), or about 2-6 mm, or about 3-4 mm, or about 4-5 mm, or other values. A more precise range may be determined according to a type of the artificial valve, which may be automatically detected (e.g., from the image), from the medical record, and/or manually entered. Optionally, during multiple iterations for multiple fluoroscopic images received in real time, the processor monitors the images to dynamically track the detected device depth (i.e., the distance between the lower plane of the patient aortic valve's cusps, and the lower plane of the implanted device).

Optionally, the indication of the depth is computed by the processor calibrating the depth according to a detected physical feature of the artificial valve prosthesis for which a fixed dimension is known. For example, diamond shaped cells of the artificial valve prosthesis which have a dimension that is known and/or fixed. The calibration is done to convert the device depth from the current image to an actual distance.

Optionally, the ML model generates a first outcome of a location of the artificial valve prosthesis and a second outcome of a location of the anatomical landmark, for example, 3D and/or 2D coordinates, and/or labels for pixels of the current image. The processor computes the depth as a distance between the first outcome and the second outcome, for example, as a Euclidean distance between the two sets of coordinates, and/or between the labelled pixels.

At 210, the processor obtains and/or computes one or more secondary outcomes. It is noted that one or more of the secondary outcomes listed below may be an outcome obtained from the ML model as described with reference to 208. The secondary outcomes may be obtained, for example, from the ML model, from one or more secondary ML models, and/or from an analysis of the images (e.g., by image processing code).

In some embodiments, the secondary outcomes are computed relative to the annulus line determined as described with reference to 208. Alternatively, in some embodiments, the secondary outcomes are computed without determining the annulus line as described with reference to 208.

Alternatively or additionally, the secondary outcome is an orientation of a heart in which the valve prosthesis is being implanted. The orientation may be computed by determining an annulus line of the heart by analysing the at least one fluoroscopic image, and computing the orientation relative to the heart. The annulus line may be used to determine if the heart is horizontal.

Alternatively or additionally, the secondary outcome is an opening percentage of the aortic valve prosthesis device. When the opening percentage is below a threshold (e.g., 80%), the operator may revert back, retract and close the prosthetic valve, and reposition the prosthetic valve. The opening percentage may be computed by analysing the aortic valve prosthesis in the at least one fluoroscopic image (e.g., measuring diameter and/or feeding into a secondary ML model), and/or by comparing a diameter of the aortic valve prosthesis to previously obtained fluoroscopic images to determine an amount of increase in the diameter.

Alternatively or additionally, the secondary outcome is the annulus line of the heart. The annulus line for the at least one fluoroscopic image may be computed from two or more previously acquired images at different projections, for example, by computing a transformation from the previously acquired images to the current image, and computing the annulus line based on the transformation. For example, the processor uses the pre planning CT (or other previously acquired image) and current real time fluoroscopy image, and the location of the annulus line at a known projection. These inputs are used for extracting the 3D transformation between subject's pre planning CT anatomy and the real time position. Using this transformation, the processor may draw the annulus line in different fluoroscopy projections, for example, by feeding the new fluoroscopic projection angulation into to a transformer.

Alternatively or additionally, the secondary outcome is the end location of the aortic valve prosthesis device relative to a location of a native valve of the subject. The end location and/or distance relative to the native valve may be computed by analyzing the image(s), by image processing code and/or a secondary ML model, to detect the end of the prosthetic valve and location of the native valve, and computing the difference therebetween. Optionally, maximal distance and minimal distance of the end location are computed by analyzing historical values of the distance of the end locations determined for preceding images.

Alternatively or additionally, the secondary outcome includes one or more of a proximal location of the aortic valve prosthesis device relative to the native valve location, and/or the proximal location of the aortic valve prosthesis device relative to the location of the coronaries' orifices.

Alternatively or additionally, the secondary outcome includes an angle, i.e. tilt, of the aortic valve prosthesis device relative to the native valve orientation.

Alternatively or additionally, the secondary outcome includes a distal end width of the aortic valve prosthesis device.

Alternatively or additionally, the secondary outcome includes a maximal width of the aortic valve prosthesis device, for example, based on historical values of preceding widths computed from preceding images.

Alternatively or additionally, the secondary outcome includes a maximal length of the aortic valve prosthesis device, for example, based on historical values of preceding lengths computed from preceding images.

Alternatively or additionally, the secondary outcome includes a computation of the curvature of the guidewire.

Alternatively or additionally, the secondary outcome includes an automatically detected change in projection from a previously captured image that excludes the artificial valve prosthesis (e.g., prior obtained CT scan of the subject before the procedure) to the at least one fluoroscopic image for one or more predefined projections, for example, a cusp overlap view, and/or an all cusps view. The projection angle may be computed, and/or the projection label may be presented.

Alternatively or additionally, the secondary outcome includes a depth change of the artificial valve prosthesis while the artificial valve prosthesis is partially or fully opened, and while the artificial valve prosthesis is being dragged downwards towards the heart and/or upwards away from the heart,

Alternatively or additionally, the secondary outcome includes an indication of whether a location of the balloon is too deep or too high.

Other computations described herein, such as related to the device depth, width, tilt, and/or guidewire curvature, may be secondary outcomes.

At 212, the processor may present the indication and/or secondary indication as a visual overlay over the at least one fluoroscopic image on a display. Alternatively or additionally, the indication and/or secondary indication may be an adaptation of the at least one fluoroscopic image.

Alternatively or additionally, the indication and/or secondary indication may be an alert, for example, an audio message played on speakers, a text message, a pop-up box, an arrow, and the like, which may be presented on the fluoroscopic image(s) and/or on another display.

At 214, a new record may be formed from the fluoroscopic image(s) and/or the outcomes generated by the ML model(s) and/or the secondary outcome(s). The new record may be used for dynamically updating the ML model(s), for example, the ML model(s) is a generic model trained on records of multiple different subjects and/or trained on a generic model (e.g., rendered as an aggregation of multiple images, a non-human simulation, and the like). The generic model may include a generic artificial aortic valve prosthesis. The new record is a form of transfer learning for customizing the generic ML model for the subject. For example, the ML model may be trained a-priori on the generic model to learn the shapes of the aortic valve's cusps and/or the shape of the artificial aortic valve prosthesis and/or a shape of calcification presence in the heart (also referred as signature of calcification). During the procedure, the generic ML model may be fine-tuned and/or a transfer learning approach may be used, on the actual aorta shape of the specific patient or the signature of calcification of the specific patient, by dynamic training of ML model using training records created from images taken from the specific patient (e.g., in several breathing cycles of the patient, e.g., 1-3 cycles) of the chest fluoroscopy including the area of aortic valve.

In some embodiments, the generic ML model may be fine-tuned and/or a transfer learning approach may be used, on the actual signature of calcification of the specific patient obtained from pre-procedure CT image of the patient and/or fluoroscopic images taken when the patient was injected with contrast.

In some embodiments, the ML model may learn the relation, e.g., spatial relationship between the annulus line and the signature of calcification of the specific patient.

Optionally, the new record includes a fluoroscopic image of the subject in which contrast has been injected, optionally labelled with one or more of the indications as ground truth. The ML model may be dynamically updated with the new record, which improves inference performance of the ML model on additional fluoroscopic images of the subject without contrast. The ML model may learns the specific anatomy of the subject (e.g., the signature of calcification of the specific patient) from the contrast enhanced images to apply to non-contrast enhanced images.

Optionally, the ML model is a generic ML model. The processor dynamically updates the generic ML model to create a customized ML model for the subject by dynamically creating customized records that include fluoroscopic images of the subject, and dynamically training the generic ML model on the customized records. The customized records may include a first set of fluoroscopic images of the subject with contrast and a second set of fluoroscopic images without contrast. Ground truth labels are applied to the customized records that include the first set of fluoroscopic images with contrast, to mark a target anatomical feature (e.g., the signature of calcification of the specific patient). The processor generates a mask for the first set of fluoroscopic images with contrast to mask pixels of a target anatomical features, for example, mask a region(s) depicting the aorta and aortic valve. The processor replaces the masked region with spatially corresponding pixels of the region from the second set of fluoroscopic images without contrast to generate a third set of synthesized images. The processor assigns the ground truth labels of the first set to the third set of synthesized images, for creating a third set of records that include the third set with ground truth label. The processor trains the ML model on the third set for detecting the target anatomical feature, i.e., the indication (e.g., aortic plane line) on images without contrast.

In other words, the ML model may “learn” to associate the label of the annulus line of the subject.

Alternatively or additionally to the masking process, the target anatomical feature may be redrawn (e.g., by image processing code) over a plurality of respiratory and/or cardiac cycles with matching frames of the second set of fluoroscopic images without contrast to create a fourth set of training records. The ML model may be self-trained to use the non-contrast images of the fourth set for detecting the target anatomical feature (e.g., aortic plane line).

Optionally, images are processed to exclude pixels of a region of the ascending aorta and optionally a “halo” around the region, for example, 2-6 mm, or about 5 mm around the region.

The ML model may use the learning of the labelling of the aortic plane (and/or aortic frame) independently of the angiogram.

At 216, the subject may be treated based on the alerts and/or visual indications, for example, by deploying the prosthetic aortic valve in the identified correct location such as within the target depth, moving the valve to a different position, retracting the valve, adjusting the guidewire, and the like. In some embodiment, the physician may implant the prosthetic aortic valve based on the indication of the annulus line.

At 218, one or more features described with reference to 202-216 are dynamically iterated during the procedure, for guiding the valve prosthesis to the correct location for deployment and/or for assisting the physician in guiding the valve prosthesis to the correct location for deployment.

For example, the processor may identify and/or alert the operator when a target device depth (e.g., within a target range) is obtained, and/or a recommendation to the user where to deploy the valve prosthesis may be generated. Alternatively or additionally, prior to implantation of the valve prosthesis, the process may detect the annulus line and/or direct the marker of the artificial valve prosthesis to be at the same level.

The features are iterated while the valve is transported via the descending aorta and/or ascending aorta on its way to be implanted in the native aortic annulus, for assisting the operating with real time navigation of the valve.

Referring now back to FIG. 3, it is noted that some features of the training are described herein, for example, with reference to FIGS. 2, 9, 11, 12, and 14. Those features may also fit into features described with reference to FIG. 3, but are omitted for clarity of reading, to avoid duplication.

At 302, one or more fluoroscopic images are obtained. The fluoroscopic image(s) depict an aortic valve prosthesis device in an aorta of the subject. The image may exclude injected contrast and/or may include injected contrast. At least some images include injected contrast.

Optionally, images of the subject acquired prior to the procedure that exclude the aortic valve prosthesis are obtained, for example, from 2D x-rays and/or a CT scan of the subject, such as done for pre-procedure planning.

Optionally, the images depict a guidewire, for example, for training an ML model to detect guidewire curvature.

Optionally, the image depict an aortic valve, for example, for training an ML model to detect device depth, and/or tilt, and/or width.

At 304, the image may be processed. Processing may be done as described herein, for example, with reference to 205 of FIG. 2, for example, extracting one or more anatomical landmarks or structures. For example, template of calcium signature of the subject, tissue thickening and/or blood flow derived dynamic changes.

Other processing may be done, for example, detecting the distal end of a partially expanded THV as described with reference to 1506 of FIG. 11, detecting the guidewire as described with reference to 1604 of FIG. 12, and/or detecting the aortic valve prosthesis as described with reference to 1804 of FIG. 14.

At 306, one or more ground truth labels are defined. Ground truth labels may be defined, for example, manually by a user (e.g., placing labels and/or markings on the image via a graphical user interface), automatically by code (e.g., image processing code that automatically identifies certain features), and/or by correlating with another image, for example, labels on a contrast enhanced image are mapped to corresponding locations on a non-contrast enhanced image, where the contrast enhanced image and the non-contrast enhanced image are correlated to each other such as via common key points.

Optionally, the ground truth label is an indication of the annulus line of the heart of the subject, middle of a subject's native aortic valve, top of a subject's native aortic valve, one or more commissures of a subject's native aortic valve, the leaflets of a subject's native aortic valve and the sinuses next to a subject's native aortic valve.

Other ground truth labels described herein may be used, for example, curvature of the guidewire, device depth, tilt, and/or width.

Optionally, such indication may guide the physician for a correct implantation for implanting the aortic valve prosthesis device in the aorta.

At 308, a training record is created. The training record includes the image(s), optionally processed such as the extracted guidewire and/or THV and/or the features extracted from the image, and may include the ground truth labels when available.

At 310, features described with reference to 302-308 are iterated for creating multiple records.

At 312, one or more training datasets of multiple training records are created.

At 314, one or more ML models are trained on the training dataset(s).

Referring now back to FIG. 4, fluoroscopic image 602 depicts an artificial prosthetic valve 610 for implantation. Line 604, which is automatically generated as an outcome of the ML model in response to an input of image 602, is generated as an overlay of image 602. Line 604 indicates a location of the annulus (i.e., lower plane of the subject's aortic valve cusps). Metrics 606, for example, device depth and device width, are computed based on indications outputted by the ML model, as described herein. Metrics 608 may be, for example, a plot along time indicating variations in values of the width of the device (i.e., the artificial prosthetic valve) as the operator expands and/or retracts the artificial prosthetic valve, and/or depth of the device (i.e., the artificial prosthetic valve) as the operator moves the artificial prosthetic valve proximally and/or distally.

Referring now back to FIG. 5, fluoroscopic image 702 depicts an artificial prosthetic valve 710 for implantation, during contrast injection, and in a more expanded state than valve 610 of FIG. 4. Line 704, which is automatically generated as an outcome of the ML model in response to an input of image 702, is generated as an overlay of image 702. Line 704 indicates a location of the annulus (i.e., lower plane of the subject's aortic valve cusps). Metrics 706, for example, device depth and device width, are computed based on indications outputted by the ML model, as described herein. Metrics 708 may be, for example, a plot along time indicating variations in values of the width of the device (i.e., the artificial prosthetic valve) as the operator expands and/or retracts the artificial prosthetic valve, and/or depth of the device (i.e., the artificial prosthetic valve) as the operator moves the artificial prosthetic valve proximally and/or distally.

Referring now back to FIG. 6, fluoroscopic image 802 depicts an artificial prosthetic valve 810 for implantation. Detected lower plane of the subject aortic valve's cusps 804 and detected lower plane of the prosthetic device 806 are shown. Features 804 and/or 806 may be computed by the ML model, optionally two ML models using the dual path processing, as described herein. The device depth is computed as the difference in distance between features 804 and 806, as described herein.

Reference is also made to FIG. 7, which is a flowchart of a method of tracking an anatomical structure of a subject, in accordance with some embodiments of the present invention. The method enables tracking the anatomical structures using images without injected contrast.

Features described with reference to FIG. 7 may be implemented with, replaced by, combined with, and/or used in addition to, other features of other methods described herein.

At 902, a contrast image (also referred to as a contrast enhanced image) that includes injected contrast, is analyzed to detect the anatomical structure. The anatomical structure may be an annulus of a native valve, optionally an aortic valve. The contrast image may depict the aortic root and surrounding tissues. The contrast may be injected within the aortic root, depicting the contour of the annulus. For example, the contour of the annulus may be the boundary between the bottom of the contrast filled aorta and the surrounding tissues and/or left ventricle without contrast.

The annulus may be automatically detected by detecting the contour of the annulus, which may include two or more cusps of the native valve. The nadirs of the two or more cusps may be automatically identified. The annulus, also referred to as the annulus line, may be defined as the line joining the two or more nadirs.

Alternatively or additionally, a 3D image of the subject (e.g., CT, MRI) which may be obtained prior to the procedure, which depicts an annulus plane and/or cusps and/or nadir of cusps, may be projected to a 2D plane according to a pose of the image sensor that captured the contrast image. The 2D plane may be registered to the contrast image. The annulus line of the 2D plane may be mapped to the registered contrast image.

The contrast images and/or non-contrast images may be 2D images, such as fluoroscopic images.

The contrast image may be automatically detected by optical based approaches, for example, a video icon indicating contrast injection appearing on the 2D images (e.g., fluoroscopic images) may be detected.

The computed annulus line may be presented on the contrast image. Optionally, an operator (e.g., the physician) may confirm the location of the annulus line, and/or correct the location. Optionally, the method may include requesting the operator (e.g., the physician) to confirm the location of the annulus line, e.g., by a pop-up on the screen.

At 904, a spatial relationship between the annulus line and a detected fiducial object (detected in the same image used to detect the annulus line) may be computed. The fiducial object may be, for example, an accessory catheter located in proximity to the native valve, such as a pigtail catheter that may be used for the injection of contrast. Alternatively or additionally, the fiducial object comprises one or more anatomical features of the subject, for example, a feature of the native aortic valve, a feature of the left ventricular outflow tract (LVOT), a feature of a coronary artery. The anatomical feature(s) may be detected, for example, by a trained ML model.

The spatial relationship may be computed between the annulus and a center of mass of the fiducial object, e.g., the accessory catheter.

The spatial relationship may be, for example, one or more distances (e.g., in pixels and/or millimeters), and/or one or more vectors that indicate relative direction, and/or a geometric shape of a defined size (e.g., triangle having a vertex at the center of mass and a base of the annulus line), and/or a mapping (such as a mapping function).

Alternatively or additionally, the spatial relationship may be computed by projecting the fiducial object to the structure, optionally the annulus line.

At 906, the anatomical structure, optionally the annulus line, is automatically detected by analyzing a target image that excludes injected contrast (also referred to herein as a non-contrast image). The target image that excludes injected contrast may be captured after the contrast image that depicts contrast was captured, i.e., after the contrast has dispersed.

The anatomical structure, optionally the annulus line, may be automatically detected by detecting the fiducial object within the target image that excludes contrast, and applying the spatial relationship. For example, the center of mass of the fiducial object is computed from the target image, and a vector implementation of the spatial relationship is used to map the location of the center of mass to the annulus line.

At 908, the detected anatomical structure is presented. Optionally, the detected annulus is presented on a display. The detected annulus may be presented as a visual overlay over the target image on a display.

At 910, one or more features described with reference to 906 and 908 may be iterated, for dynamically tracking the location of the anatomical structure, for example the annulus line, on subsequent non-contrast images. The tracked annulus line may be used, for example, for guiding location of deployment of a prosthesis heart valve.

Optionally, the annulus line, detected as described with reference to 902, represents the annulus line prior to an event that disrupts the annulus, for example, inflation of a balloon within the native valve (e.g., valvuloplasty) such as to crack calcifications and/or adhesions to increase the area of the native valve to enable deployment of the prosthetic heart valve. The annulus line may be detected after the event, using the spatial relationship computed prior to the event, as described with reference to 906. The annulus line may be detected after the event using the same spatial relationship computed prior to the event, since the event disrupts the leaflets and/or calcifications adhering to the leaflets and surrounding tissues, but the annulus line remains substantially unaffected.

Referring now back to FIG. 8, image 1002 (e.g., fluoroscopic images) depicts annulus line 1004, fiducial object 1006, and spatial relationship 1008. Annulus line 1004 is computed as described herein, for example, a line drawn between a first nadir of a first cusp 1010A and a second nadir of a second cusp 1010B of the aortic valve. Fiducial object 1006 may be for example, a pig tail catheter. The center of mass of the catheter may be computed and used. Spatial relationship 1008 is computed, for example, as a triangle between the center of mass of fiducial object 1006 and annulus line 1004.

Image 1002 is shown with contrast 1012 within the aortic root. Once spatial relationship 1008 is computed, location of annulus line 1004 may be computed using fiducial object 1006 for images that exclude contrast 1012, where annulus line 1004 cannot be directly visualized.

Referring now back to FIG. 11, the flowchart of the process for computing the distance between the annulus line and the distal end of the THV, is described.

At 1502, a target image, optionally non-contrast, optionally 2D, is accessed. The image may be a fluoroscopic image obtained during a TAVI procedure.

The target image may depict a partial expansion of a transcatheter heart valve (THV) positioned within the native aortic valve of the subject.

At 1504, an annulus line (also referred to herein as a lower annulus) of the subject's native aortic valve may be automatically detected by analyzing the target image.

Various approaches may be used for detecting the annulus line, for example, as described herein, such as with reference to 208 of FIG. 2, FIG. 7, FIG. 9, and FIG. 10. Other exemplary approaches are now described.

Optionally, a candidate location of the lower annulus of the native aortic valve is detected by analyzing sample image(s) depicting the lower annulus of a native aortic valve that excludes presence of the THV within the native aortic valve. The sample image(s) may include contrast. The candidate location may be detected and/or learned from the sample image(s) of the subject obtained prior to placing the THV in the native aortic valve. Alternatively or additionally, the candidate location is learned from sample image(s) of sample individual(s) and/or of a generic model. The sample image may be registered with the target image (which depicts the partially expanded THV within the native aortic valve). The candidate location of the sample image(s) may be mapped to the target image to obtain the lower annulus of the target image. The mapping may be performed by computing a transformation from the sample image to the target image, and applying the transformation to the candidate location for obtaining the lower annulus. The sample image(s) may include injected contrast located within the cusps of the aortic valve and the ascending aorta. The target image may exclude injected contrast within the cusps of the aortic valve and the ascending aorta. The lower annulus detected in the sample image(s) may be mapped to the target image for identifying the lower annulus in the target image.

At 1506, a distal end of the partially expanded THV may be automatically detected by analyzing the target image. The analysis may be performed by feeding the target image into a machine learning model trained on a training dataset of records, where each record includes a sample image of the partially expanded THV, and a ground truth indicating the distal end thereof. In another example, the analysis may be performed by image processing and/or computer vision approaches, for example, comparing the target image to a template depicting the distal end of the partially expanded THV. A correlation may be computed between the target image and the template. The distal end of the partially expanded THV may be detected when the correlation is above a threshold.

Exemplary details of ML models for detecting the distal end of the partially expanded THV and/or of the lower annulus are now described.

Optionally, the ML model is trained on a training dataset of records, where each record includes a sample image of a sample individual or of a generic model labelled with a ground truth label indicating a lower annulus of a native aortic valve and/or a distal end of a partial expanded THV for positioning within the native aortic valve. The ML model may be dynamically further trained on images of a subject depicting the lower annulus of the native aortic valve, to obtain a personalized ML model. The target image of the subject may be dynamically fed into the personalized ML model to obtain a detected lower annulus and a detected distal end of the partially expanded THV.

Optionally, the annulus line may be detected independently of the THV (e.g., as described herein). The THV may be detected using other approaches, for example, using an ML model, image processing, and/or computer vision based approaches. For example, the ML model for detecting the THV may be training on a training dataset of records, where each record includes a sample image of a sample THV, optionally in a compressed state and/or in various states of deployment (e.g., partial deployment, full deployment). In another example, image processing and/or computer vision based approaches used to detect the THV include for example, detecting an edge of the THV, detecting a pattern of pixel intensity values unique to the THV, and/or correlating with a template depicting the THV.

Optionally, a record(s) used to train the ML model include multiple images captured over multiple phases of a cardiac cycle and/or a breathing cycle of the subject.

Optionally, a personalized mask excluding a region around the lower annulus and/or the distal end of the partially expanded THV is computed. The mask may be applied to the sample image of the record used to train the ML model and/or to the target image(s) fed into the ML model. The mask may remove irrelevant data from the image(s) which may help the ML model to avoid focusing on irrelevant features.

Optionally, contrast enhanced image(s) of the subject depicting injected contrast located within the cusps of the aortic valve and the ascending aorta, are automatically analyzed to identify the lower annulus, for example, as a contour of the contrast and/or using ML model approaches, and/or other approaches described herein. The identified lower annulus may be automatically labelled in the contrast enhanced image(s). A mask over the contrast enhanced region of the contrast enhanced image that includes the cusps and/or the ascending aorta may be applied to define a masked region. A synthesized image may be created by replacing the masked region of the contrast enhanced region with values of pixels corresponding to the masked region in a non-contrast enhanced image. The record including the synthesized image labeled with the identified lower annulus may be used for training the ML model for detecting the lower annulus in response to an input of a non-contrast enhanced image.

At 1508, the device depth of the THV is computed. The device depth is a distance between the lower annulus and the distal end of the THV.

The term device depth and distance are used interchangeably.

Optionally, the detected lower annulus is represented as a first line representing a plane of the lower annulus. The detected distal end of the THV is represented as a second line representing a plane of the distal end of partially expanded THV. The distance is computed between the first line and the second line.

Alternatively or additionally, a fiducial marker of the THV having a known dimension(s) is automatically detected. A calibrated distance is computed by calibrating the distance according to the known dimension. The fiducial marker may include, for example, a strut or portion thereof of a scaffold of the THV that remains the known fixed dimension throughout expansion of the THV. In another example, the fiducial marker may include a catheter (pigtail used to inject the contrast) located in the aorta have a fixed dimension such as of a marker.

At 1510, an automatic determination may be made whether the distance is within a target range indicating correct positioning of the THV within the native aortic annulus. The target range may be defined according to an identifier of the THV. For example, different THV may have different identifiers and different target ranges.

At 1512, one or more data items may be provided, optionally presented on a display, optionally within a graphical user interface (GUI). For example, the distance and/or the determination may be presented.

Optionally, an overlay over the target image that visually marks the lower annulus and/or the distal end may be provided.

Optionally, a visual and/or an audio indication of when the distance is within the target range may be presented. The visual indication may be presented, for example, in the form of a binary or tertiary indication, such as three colors. A first color indicates that the distance is within the target range. A second color indicates that the distance is external to the target range, optionally in a certain direction. For example, that the distance is lower than the target range. A third color may indicate that the distance is external to the target range in the other direction, for example, that the distance is greater than the target range. In another example, a coded bar is presented, representing the possible values of the distance, from low to higher. An indication on the coded bar represents the current distance. A region of the coded bar may indicate the target range.

Optionally, when no distal end of the partially expanded THV is detected, a visual and/or an audio indication of where to position a distal end of the THV in the compressed state for expansion of the THV such that the distal end of the THV when expanded is predicted to be located within the target range, may be presented.

At 1514, when the distance is external to the target range, the operator may be instructed to move the THV to another location.

At 1516, one or more features described with reference to 1502 to 1514 are iterated for tracking the device depth.

Optionally, when the THV is a self-expanding THV, and the distance is monitored over sequential target images during the self-expansion of the THV.

Optionally, the candidate location and the lower annulus are detected and tracked over sequential sample images and target images by matching phases of breathing cycle(s) and/or at least one cardiac cycle(s) depicted in the sample images to corresponding phases of the target images.

At 1518, when the distance is within the target range, the THV may be deployed, for example, for treating subject for a dysfunctional aortic valve by implanting the THV within the native aortic valve.

Additional features described below may be implemented with embodiments described above, for example, as described with reference to FIGS. 1-17C.

An aspect of the present invention includes a method of guiding a trans-catheter aortic valve replacement intervention in a patient.

Trans-catheter aortic valve replacement (TAVR) is a minimally invasive heart procedure to replace a thickened aortic valve that can't fully open, a condition known as aortic valve stenosis. The aortic valve is located between the left ventricle and the aorta. If the valve doesn't open correctly, blood flow from the heart to the body is reduced.

TAVR can help restore blood flow and reduce the signs and symptoms of aortic valve stenosis—such as chest pain, shortness of breath, fainting and fatigue.

Trans-catheter aortic valve replacement may also be called trans-catheter aortic valve implantation (TAVI).

As used herein, guiding an intervention may include providing information about the manner at which the intervention proceeds. For example, the information may include images of the interior of the patient's body, where intervention takes place or where a device used by the operating surgeon is navigating. The information may be provided to the operating surgeon or to any other member of the operating stuff.

The disclosed method can be implemented only on a computer. The computer typically includes a processor and a digital memory that stores instructions, that when executed by the processor cause the processor to carry out a method as described herein. In some embodiments, the computer may also include a display for displaying information for guiding the intervention.

The method includes obtaining fluoroscopic 2D images capturing an aortic valve prosthetic device in the aorta of the patient, e.g., in the descending aorta of the patient. The prosthetic device may be, for example, Evolute™ by Medtronic, Porticco™ by Abbott, or LOTUS Edge™ or NEO™ by Boston Scientific.

These and other aortic valve prosthetic devices have to be oriented in alignment with the native commissure of the native heart valve, and include markers that should help the operating surgeon implanting the prosthetic device at the correct orientation. In some of the devices the marker includes a plurality of marking units, e.g., radiopaque dots or short lines, that are oriented one in respect to the other in some predetermined manner when, and only when, the device is properly oriented.

However, the markers are frequently hard to find in the image, and the operating surgeon is required to invest considerable cognitive resources for finding it in the image, and determining the state of the marker, which is indicative to the orientation being proper or not.

It is noted that the appearance of the marker in the 2D image depends not only on the way the marker is aligned in respect to the patient, but also on the positioning of the imager. Therefore, it is not necessarily sufficient to identify how the marker appears, but also the viewing angle at the image was taken. Usually, the implantation of the prosthetic valve device is carried out in a certain, preferable, fluoroscopic view (i.e., “cusp overlap view”).

In some embodiments, if the view is identified not to be the preferable (e.g., cusp overlap) view, the methods or apparatuses alert that the view is not the preferable one. In some embodiments, an indication of the of the annulus line of the heart of the subject also be provided even if the image is obtained with an imager at non-preferable position.

The fluoroscopic images may be obtained from an imager that is integral to the guiding device, or from an independent imager. In some embodiments, the guiding computer is directly connected to the imaging device, and receives as input data from the imaging device. Optionally, these data are the same used by the imager for producing and displaying the fluoroscopic image on the imager display. In some embodiments, the computer may include (or may receive input from) a camera that photographs the imaging device display, and the 2D images are obtained from this camera for purpose of the guiding.

The fluoroscopic images are preferably obtained and processed online, so that the operating surgeon may receive the guidance in real time, although in some embodiments, the guiding may be provided after the fact, for post hock analysis and staff education.

At least one of the obtained fluoroscopic images is fed to a machine-learning model trained to identify the annulus line of the heart of the subject and output an indication to of the annulus line of the heart of the subject.

In some embodiments, the training is with images labeled by human experts. The labeling includes a label of the position of the annulus line of the heart of the subject. In some embodiments, the machine-learning includes two modules, one trained to identify the template of calcium signature of the subject in the image, and another trained to identify the of the annulus line of the heart of the subject. Calcium signature may also refer to herein as calcification signature.

The images used for training may include images received from the imager, and in addition, the same images transformed randomly for rotation, zoom, and/or shift. In some embodiments, each image is transformed for up to 100 additional times, so with 300 images the training is actually happening on 30 000 slightly different images which may decrease the risk of overfitting. This way the amount of the images used in the training may be increased without having to collect and label addition images. Optionally, the training is done using stochastic optimization, and each epoch a different set of the images is used in the training.

The output from the machine-learning model may include indication to the position of the annulus line of the heart of the subject in the image The output may be received by a computer module that controls a display to indicate the position of the annulus line of the heart of the subject, for example: the annulus line may be highlighted on the image; or otherwise marked as an overlay over the image. In some embodiments, the display may be visual. e.g., a textual message may appear on the display, or an indicator may be lighted with lights of different colors, etc.

In some embodiments, the method is repeated with new images obtained from the imager as the operation proceeds. In such embodiments, from time to time (e.g., every 0.2, 0.5, 1, 2 or 5 seconds), a new image is fed into the machine-learning model to detect the annulus line or another structure of a native valve. The annulus line may be displayed to an operator continuously during the procedure. This may enable to physician to implant the valve prosthesis device at the correct location.

Referring now back to FIG. 12, features described with reference to FIG. 12 may be implemented by components of system 100 described with reference to FIG. 1.

At 1602, one or more images (e.g., 2D and/or fluoroscopic images) depicting a guidewire with a body of a subject are accessed. The guidewire may be located within blood vessels and/or organs, for example, within the aorta and/or left ventricle. Additional details and/or examples of accessing fluoroscopic images are provided, for example, as described herein, such as with reference to 204 of FIG. 2.

The guidewire located in the body usually has one or more local curvatures due to the tortuous anatomical arrangement of blood vessels.

Optionally, the guidewire is a stiff guidewire. The stiff guidewire may pose a risk to a patient. For example, a tight curve (e.g., small radius below a threshold) may apply high pressure to a wall of the aorta and/or left ventricle, which may damage tissues, such as damage the conduction system of the heart.

The guidewire may be the guidewire used for navigation and/or deployment of the aortic valve prosthetic device.

Optionally, each frame of a video stream generated by a fluoroscopy imaging system is grabbed. The frame represents the fluoroscopic image described herein. Alternatively, every few frames may be grabbed. The grabbing may be performed using an off the shelf video grabber which is connected to a standard video output of the fluoroscopy imaging system. The video grabber may be connected to an integration component that transfers the raw frame (i.e., “as is”) to a work station for further analysis.

At 1604, the fluoroscopic image may be analyzed to identify the guidewire with local curvature. The guidewire may be identified by image processing means and/or ML means. The guidewire may be segmented from the fluoroscopic image. For example, as pixel values for a boundary box define the stiff wire over the fluoroscopy image, as a border delineating the boundaries of the guidewire, and/or as a mask over the fluoroscopic image that retains the guidewire and blocking out pixels of background and other details (e.g., a value of 1 indicates the aortic valve prosthesis device, and a value of 0 indicates background).

The fluoroscopic image may be analyzed by being fed into a machine learning module, e.g., guidewire detector ML model. The guidewire detector ML model may be trained for segmenting the guidewire within the fluoroscopic image

The guidewire detector ML model may be trained on a training dataset of sample fluoroscopic images depicting sample guidewires optionally with different amounts of local curvature. Each image is labelled with a ground truth segmentation of the sample guidewire.

When the guidewire is highly visible in the image, a user may easily manually mark it on sample images to create the ground truth. Alternatively, the guidewire may be automatically annotated by a running process. Overlay margin (e.g., of about 1.5 mm) may be presented on a display, for example, using dotted line. A human marking ground truth may indicate if they agree with the automatic annotation. The automatically generated ground truth may be used, for example, when the majority vote of the human markers approve the automatically annotated line is within the clinical margin. A minimal expected performance may be an agreement of, for example, at least 85%.

The guidewire detector ML model may be implemented, for example, by two cascading neural networks (NN). The first NN may be based on the ResNet50 architecture. The first NN may identify an Area of Interest (AoI). The second NN may be based on the Unet architecture. The second NN may performs a segmentation on the AoI, returning the segmented wire.

At 1606, a local curvature of the guidewire depicted in the fluoroscopic image may be computed. The local curvature may be computed using the segmentation of the guidewire. The local curvature may be computed over the whole guidewire depicted in the segmentation of the image.

The local curvature may be computed using a numeric computation approach, by computing a local radius of curvature over the segmented wire. For example, a circle is fitted to the local curvature. The local curvature may represent an arc of the fitted circle. The radius of the fitted circle is obtained as representing the local curvature.

The curvature may vary along the guidewire. Multiple different circles of different radii may be fitted to different parts of the guidewire.

The curvature may be calculated over the whole segmented guidewire, locally on points sampled from the segmentation. A process for computation of the radius of curvature may be based on a merger approach using the sampled points.

There may be multiple local curvatures, in which case, the curvature of each local curvature may be computed.

The curvature may indicate the amount by which a curve deviates from being a straight line. The curvature at a point of a differentiable curve is the curvature of its osculating circle, which is the circle that best approximates the curve near this point. The curvature of a straight line is zero. The curvature at a point may be a scalar quantity, that is, it is expressed by a single real number.

A higher value of the local curvature may indicate a sharper curvature, indicating higher risk of high pressure being applied to tissue.

The local curvature may be computed as a local radius. A lower value of the local radius may indicate higher curvature, indicating the higher risk of high pressure being applied to tissue. A higher value of the local radius may indicate low curvature.

Descriptions of one type of metric (e.g., local curvature, radius) are meant to be exemplary and not necessarily limiting. Other metrics representing curvature may be used.

At 1608, an indication of the local curvature may be presented on a display, for example, within a graphical user interface (GUI). In cases where there are multiple local curvatures, the curvature of each local curvature may be displayed.

The indication of the local curvature may be presented by generating an overlay over a portion of the guidewire in the fluoroscopic image depicting the location of the local curvature. The overlay may include a scale correlated with values of the curvature. For example, regions of the guidewire indicating where the local curvature is above a threshold and/or where the radius is below a threshold.

The overlay may be a mask over the image.

The overlay may be coded using a visual scale, according to an amount of the local radius of the local curvature. For example, a different coding for different values of the local radius, such as different codings for different ranges of the local curvature. For example, indicating high risk such as indicating likelihood of high pressure applied to the tissue for adversely impacting the conduction system of the heart, medium risk, and low risk.

The overlay may be a visual coding, for example, color and/or pattern. Different colors and/or different intensity of colors, and/or different patterns and/or different density of patterns (e.g., number of dots per centimeter) represent different values of the radius. For example, different colors for different ranges, and a variation in intensity of the color within each range. For example, red for high curvature portions (small radius), green for low curvature (large radius), and yellow for intermediate values.

Optionally, the local curvature is evaluated relative to a threshold (or other requirement) indicating a curvature high enough to likely to lead to an adverse event, for example, tissue damage, perforation, damage to conduction system. The indication of the local curvature may be presented when the radius is below the threshold. In this manner, the GUI generates an alert when high curvatures representing high risk are detected. The threshold may be a first set low as safety precaution, to avoid reaching a second threshold where the conduction system may be impacted.

Alternatively or additionally, the local curvature of the guidewire is evaluated to determine whether the curvature is within pre-defined thresholds, for example, indicating safe curvature. The indication may be presented when the local curvature is not within the pre-defined thresholds, indicating unsafe curvature.

It is noted that the curvature may vary along the length of the guidewire. Using the overlay to code the length of the guidewire visually indicates where the highest and/or most risky curvature is located, for example, relative to tissues of the body depicted in the image. The overlay may assist the operator in determining how to best move the high curvature to a different location and/or how to best reduce the high curvature, e.g., by reducing pressure on the guidewire.

Referring now back to FIG. 13, GUI 1702 may present fluoroscopic image 1708 with guidewire 1706. Local curvature 1704 (e.g., having a radius below a threshold) may be marked, for example, using dashed lines. Regions within local curvature 1704 may indicate the amount of curvature. For example, regions 1710 (e.g., colored black) indicate very low curvature, regions 1712 (e.g., colored red) indicate low curvature, regions 1714 (e.g., colored yellow/orange) indicate medium curvature, and region 1716 (e.g., colored white) indicates high curvature.

Referring now back to FIG. 12, at 1610, one or more features described with reference to 1602-1608 are iterated.

The iterations may be performed for multiple sequential images, for example, for monitoring the guidewire over the sequential images.

Optionally, a maximal curvature of the guidewire depicted in the sequence of fluoroscopic images is determined. The maximal curvature may be presented on the display. The maximal curvature may be used, for example, by the operator as a warning that such maximal curvature may occur, and for monitoring for preventing the maximal curvature from occurring and/or for reducing the maximal curvature if it does occur.

Optionally, a maximal curvature distance of the guidewire from the plane of the native aortic annulus plane is computed according to the monitoring of the location of the guidewire depicted in the sequence of fluoroscopic images. The maximal distance may be presented.

Optionally, the location of the local curvature of the guidewire relative to the aortic annulus is determined for individual images and/or tracked for multiple image. For example, whether the local curvature is above or below the annulus, and/or how far away the curvature is from the annulus. The indication of whether the local curvature is above or below the annulus, and/or distance from annulus, may be presented. A curvature above the native aortic annulus is not inside the heart and does not affect the conduction system, but may be provided as a warning to the operator that there is a risk of the curvature forming and/or moving to the inside of the heart where the curvature may impact the conduction system.

Pressure may be monitored by monitoring the local curvature of the guidewire, which may be indicative of a potential high pressure applied by the guidewire to the interior of the heart, which may adversely impact the conduction system of the heart. The detected curvature may be below the native aortic annulus plane, optionally within a chamber of the heart. By detecting the curvature indicating high pressure, the operator may reduce the curvature for reducing the pressure, for reducing likelihood of adversely impacting the conduction system of the heart. The detected portion of the local curvature of the guidewire may be displayed, for example, marked with a different color or pattern to distinguish from other portions of the guidewire that do not have local curvatures.

Referring now back to FIG. 14, features described with reference to FIG. 14 may be implemented by components of system 100 described with reference to FIG. 1. The width, depth, and/or tilt may be used to assist a user with optimal implantation of the aortic valve prosthesis device, for example, with reference to an aortic annulus line.

At 1802, one or more images (e.g., 2D and/or fluoroscopic images) depicting an aortic valve prosthesis device, optionally in an aorta of the subject, are accessed.

The fluoroscopic image(s) may exclude injected contrast.

Additional details and/or examples of accessing fluoroscopic images are provided, for example, as described herein, such as with reference to 204 of FIG. 2.

Optionally, each frame of a video stream generated by a fluoroscopy imaging system is grabbed. The frame represents the fluoroscopic image described herein. Alternatively, every few frames may be grabbed. The grabbing may be performed using an off the shelf video grabber which is connected to a standard video output of the fluoroscopy imaging system. The video grabber may be connected to an integration component that transfers the raw frame (i.e., “as is”) to a work station for further analysis.

At 1804, the fluoroscopic image(s) may be analyzed for identifying the aortic valve prosthesis device, optionally within the aorta. The aortic valve prosthesis device may be identified by image processing means and/or ML means. Optionally, the aortic valve prosthesis device is segmented from the fluoroscopic image. For example, as pixel values for a boundary box defining the aortic valve prosthesis device over the fluoroscopy image, as a border delineating the boundaries of the aortic valve prosthesis device, and/or as a mask over the fluoroscopic image that retains the aortic valve prosthesis device and blocking out pixels of background and other details (e.g., a value of 1 indicates the aortic valve prosthesis device, and a value of 0 indicates background).

The analysis may be performed by feeding the fluoroscopic image(s) into a machine learning module, e.g., device detector machine learning (ML) model. The device detector ML model may be trained for segmenting the aortic valve prosthesis device within the inputted fluoroscopic image(s).

The device detector ML model may be trained on a training dataset of sample fluoroscopic images depicting sample aortic valve prosthesis devices, labelled with a ground truth segmentation of the sample aortic valve prosthesis device.

The device detector ML model may be implemented, for example, based on a U-net architecture.

Alternatively or additionally, the fluoroscopic image(s) may be analyzed for determining an anatomical landmark, for example, an annulus line of the aortic valve and/or coronary ostia. The annulus line may be determined, for example, as described herein, and/or as described, for example, with reference to International Patent Application No. IB2023/062015 “SYSTEM AND METHOD FOR GUIDING TRANS-CATHETER AORTIC VALVE REPLACEMENT”, filed on Nov. 29, 2023, incorporated herein in its entirety. The coronary ostia may be determined, for example, by feeding the image into a coronary artery detector ML model. The coronary artery detector ML model may be trained on fluoroscopic images depicting the ascending aorta and/or aortic root, optionally at different viewing angles which correspond to the fluoroscopic imaging system viewing angle(s) likely to be used during the procedure. Each image is labelled with a ground truth marking the location of the coronary ostia, for example, a segmentation and/or boundary box around the coronary ostia and/or marking on the coronary ostia.

Optionally, additional processing is performed on the image and/or the segmentation. Optionally, a scale is calibrated according to a detected physical feature of the aortic valve prosthesis device for which a fixed dimension is known. The calibrated scale may be used in the measurement of the tilt and/or width and/or device distance.

At 1806, the fluoroscopic image is analyzed for identifying a distal end of the aortic valve prosthesis device. The distal end of the aortic valve prosthesis device may be identified by image processing means and/or ML means. Alternatively or additionally, the segmentation of the aortic valve prosthesis device is analyzed for identifying the distal end of the aortic valve prosthesis device.

The identified distal end may be represented by a straight line, for example, through the two or more most distal points on the aortic valve prosthesis device.

The outermost tip (e.g., two or more, but usually two on a 2D image) structure(s) of the aortic valve prosthesis device may be automatically extracted from the segmentation of the aortic valve prosthesis device. The two outermost tips are the points on the aortic valve prosthesis device that are the most extreme on the distal end of the aortic valve prosthesis device, i.e., one most extreme point on the distal end on the left side, and a second most extreme point on the distal end on the right side.

Optionally, the two or more outermost tips of the aortic valve prosthesis device may be obtained as an outcome of the segmentation of the aortic valve prosthesis device, or the image that depicts the aortic valve prosthesis device, fed into a tip detection ML model. The tip detection ML model may be trained on a training dataset of images and/or segmentations of the aortic valve prosthesis device labelled with ground truth markings indicating the locations of the outermost tip(s).

The distal line may be drawn between the two outermost tips of the aortic valve prosthesis device.

The depth and/or tilt and/or width may be calculated according to the detected two (or more) most outermost tips structure(s) of the aortic valve prosthesis device. Users may annotate the outermost implant structure tips independent from the code that identifies the tips. Ground truth lines, connecting between the two tips, may be automatically drawn based on the average of the annotating users (e.g., three). It is noted that all lines are linear, and therefore drawing the ground truth lines may be a simple mathematical task. For each image, the distance between the ground truth line and the automatically annotated line may be calculated, for example, as an average between the smallest and largest distance between the line of manually marked tips and the line between the automatically identified tips. A clinical margin indicating a match between the two lines may be selected, for example, about 1 millimeter (mm), or 1.5 mm, or 2 mm, or other values.

Optionally, pixel values indicating the distal end (e.g., distal line) are determined.

At 1808, a width of the aortic valve prosthesis device (e.g., in the aorta) may be computed.

The width may be of a distal end of the aortic valve prosthesis device. Optionally, the width is computed as a length of the distal line (i.e., the end of the segmented aortic valve prosthesis device).

The width may be of the aortic valve prosthesis device in a compressed state and/or partially expanded state and/or fully expanded state. The width is minimal in the compressed state, and increases as the aortic valve prosthesis device is expanded, until reaching a maximal value when fully expanded. Width may decrease, for example, in response to the operator retracting the aortic valve prosthesis device back into a sheath. As a result, there may be local minimum and/or maximum values of the width, which may not necessarily correspond to the extreme minimum and/or maximum width in the fully compressed and/or fully expanded states.

Alternatively or additionally, a length of the aortic valve prosthesis device, is computed. For example, from the distal line to a proximal line indicating the proximal distal end of the aortic valve prosthesis device. The length may be tracked as the width changes during expansion and/or retraction of the aortic valve prosthesis device. A maximal and/or minimal length of the aortic valve prosthesis device may be determined. Optionally, local maximal and/or minimal lengths are determined, as the aortic valve prosthesis device is expanded and retracted, which may be different than the extreme maximum and/or minimum lengths of the fully compressed and/or fully expanded states.

At 1810, a device depth of the aortic valve prosthesis device is computed. The device depth may be computed from a distal end location of the aortic valve prosthesis device to an anatomical landmark. Optionally, the device depth is computed as the distance from the distal line to the anatomical landmark. The anatomical landmark may be an annulus line of the aortic valve annulus and/or one or more coronary orifices (e.g., line drawn through the coronary orifices).

Alternatively or additionally, the device depth is computed from a proximal end location of the aortic valve prosthesis device to the anatomical landmark.

The device depth may be computed as an average distance between the distal line and the line representing the anatomical landmark. For example, an average of the shortest distance from the distal line to the annulus line and the largest distance from the distal line to the annulus line.

Deployment of the aortic valve prosthesis device at a target device depth may reduce or prevent complications of the aortic valve prosthesis device as described herein, for example, migration, dislodgement, paravalvular leaks, and reduced performance.

At 1812, a tilt of the aortic valve prosthesis device relative to the anatomical landmark may be computed. The tilt may be computed as an angle between the distal line and the annulus line.

Deployment of the aortic valve prosthesis device at a target tilt (e.g., no tilt angle, or low tilt angle) may reduce or prevent complications of the aortic valve prosthesis device as described herein, for example, migration, dislodgement, paravalvular leaks, and reduced performance.

Device tilt may be computed relative to the coronary ostia and/or commissures of the native aortic valve. The device tilt may indicate the alignment of the aortic valve prosthesis device relative to the coronary ostia, such that the coronary ostia will be non-blocked once the aortic valve prosthesis device is deployed.

At 1814, the width and/or device depth and/or width may be presented on a display, optionally with a GUI.

Optionally, an overlay over the region of the aortic valve prosthesis device of the fluoroscopic image indicating where the width of the aortic valve prosthesis device is measured, is generated. For example, the length of the distal line representing the width of the aortic valve prosthesis device is presented in the overlay.

Alternatively or additionally, an overlay of a first line indicating the anatomical landmark over the fluoroscopic image is generated. The first line may be, for example, the annulus line indicating the annulus of the native aortic valve, as described herein. Alternatively or additionally, the first line may indicate the location of the coronary ostia, for example, a line drawn through identified coronary ostia.

Alternatively or additionally, the overlay may include a second line, optionally the distal line and/or the proximal line of the aortic valve prosthesis device.

The device depth may be presented (and/or computed) as the distance between the first line and the second line. For example, one or more measurement lines may be drawn between the first line and the second line. The length of the measurement line(s) indicates the device depth, i.e., the distance between the first line and the second line. When the first line and second line are not parallel, there may be multiple measurement lines. The device depth may be computed as an aggregation of the multiple measurement lines, for example, an average of the length of the measurement lines. The value of the distance, i.e., device depth, may be presented in proximity to the measurement lines.

The tilt may be presented (and/or computed) as an angle between the first line and the second line. For example, an angle between the first line and second line is presented. The value of the angle may be presented.

The width may be presented (and/or computed) as a length of the distal line.

Optionally, the GUI indicates a target range of the device depth and/or tilt suitable for deployment of the aortic valve prosthesis device. The GUI may provide a visual indication of the tilt and/or device depth within the target range or the regions external to the target range, optionally, according to a scale correlate with values of the tilt and/or device depth and/or width. The scale may be defined by a user, preset, and/or dynamically computed such as according to possible values that may be obtained in real time. For example, regions external to the target range may be coded for indicating intensity of closeness to the target range, for example, using a gradient of color and/or pixel intensity values where more intense pixels indicate being closer to the target range. Regions within the target range may be coded for indicating intensity of accuracy, for example, using a gradient of color and/or pixel intensity values where more intense pixels indicate higher accuracy within the range.

Alternatively or additionally, a plot along time indicating variations in values of the width and/or tilt and/or device depth is dynamically updated from successive frames, for example, as the operator expands and/or retracts and/or moves the aortic valve prosthesis device proximally and/or distally and/or rotates it.

Referring now back to FIG. 15, the overlay over aortic valve prosthesis device 1904 depicted in fluoroscopic image 1906 within GUI 1902, indicates depth and/or tilt of aortic valve prosthesis device 1904. The overlay may include one or more of:
An annulus line 1908 indicating the annulus of the native aortic valve.
A distal line 1910 indicating the distal end of aortic valve prosthesis device 1904.
An angle (α) 1912 between distal line 1910 and annulus line 1908, indicating tilt.
Detected outermost tips 1914A-B (where 1914A is also marked as A, and 1914B is also marked as B) of aortic valve prosthesis device 1904. It is noted that distal line 1910 is drawn between outermost tips 1914A and 1914B.
Measurements lines 1916A-B indicating distance between annulus line 1908 and distal line 1910. The device depth is computed according to the length of measurement lines 1916A-B, for example, as an average of the two measurement lines 1916A-B respectively located at outermost tips 1914A-B. dA denotes the distance of measurement line 1916A. dB denotes the distance of measurement line 1916B. Device depth may be calculated as (dA+dB)/2.

Referring now back to FIG. 16, dashboard 2000 indicates device depth 2002 and/or tilt 2004. Device depth 2002 may be presented visually, for example, an indication along a scale and/or coded bar, where a target region 2006A (e.g., colored green) indicates the device depth is within a target suitable for deployment, a hazard region 2006B (e.g., colored red) indicating the device depth is outside the target and the aortic valve prosthesis device should not be deployed, and an intermediate region 2006C (e.g., colored yellow) indicating an intermediate state of close to the target but not within the target. Pixels within respective regions 2006A-C may be presented with varying intensity, where a more intense pixel value indicates further away from the target and a less intense pixel intensity indicates closer to the target. A numerical value of the device depth may be computed. Tilt 2004 may be presented for one or more axes and/or degrees of freedom, for example, angular rotation and/or up/down tilt. Tilt 2004 may be presented using a dial and/or coded bar (curved as shown and/or straight).

The scale and/or boded bar and/or dial may be selected and/or set by a user. For example, the user may select colors and/or other visual indications (e.g., pattern) for each region. In case of no selection by a user, numerical values may be presented for device depth and/or tilt.

Referring now back to FIGS. 17A-C, exemplary GUI 1902 (as described with reference to FIG. 15) that includes an overlay and dashboard 2000 (as described with reference to FIG. 16) during various stages of a procedure for implanting aortic valve prosthesis device 1904. FIG. 17A is captured before deployment of 1904 aortic valve prosthesis device. FIG. 17B is captured during deployment. FIG. 17C is captured after deployment. The overlay presented within GUI 1902 includes annulus line 1908, distal line 1910, and detected outermost tips 1914A-B which are the end points of distal line 1910, as described with reference to FIG. 15. Dashboard 2000 indicates depth and/or tilt during the different stages of deployment of aortic valve prosthesis device being expanded and/or maneuvered for implantation in the aortic valve, as depicted in FIGS. 17A-C. It is noted that in FIG. 17C, the aortic valve prosthesis device has not been adequately deployed, since dashboard 2000 indicates that the device depth 2002 and/or device tilt 2004 are not optimal, since the arrows of dashboard 2000 point to the intermediate regions rather than to the target region, as explained with reference to FIG. 16.

Referring now back to FIG. 14, at 1816, one or more features described with reference to 1802-1814 are iterated. The width and/or tilt and/or device depth may be monitored over multiple sequential images during the iterations.

Optionally, the width and/or tilt and/or device depth are monitored to determine whether the width and/or tilt and/or device depth are within a target range, or deviate from the target range. An alert may be generated in response to detecting deviation from the target range, for example, a pop-up is presented within GUI, an audio alert is generated, and/or the dashboard and/or overlay are updated with a unique marking to indicate deviation (e.g., color, bolding, pattern).

Optionally, the width and/or tilt and/or device depth are monitored for computing a maximal and/or minimal values, for example, during expansion and/or retraction of the aortic valve prosthesis device. The maximal and/or minimal values may be presented on the display.

Optionally, local maximal and/or minimal width and/or tilt and/or device depth are determined, as the aortic valve prosthesis device is expanded and retracted, which may be different than the extreme maximum and/or minimum width and/or tilt and/or device depth of the fully compressed and/or fully expanded states. The local minimum and/or maximum values may be presented on the display.

Referring now back to FIG. 9, features described with reference to FIG. 9 may be implemented for detecting and/or tracking the annulus line, which may be used for computing the width and/or tilt of the THV relative to the annulus line, as described herein. The features described with reference to FIG. 9 may be implemented with, replaced by, combined with, and/or used in addition to, other features of other methods described herein.

At 1104, 2D image(s) (e.g., fluoroscopic image(s)) of the subject (also referred to herein as target images) may be taken when the subject is injected with contrast during the intervention. The contrast may injected into the aortic root, such as during a TAVI procedure.

The target image depicting contrast may be referred to herein as a contrast enhanced image.

The target image may depict a valve prosthesis device in the subject. The valve prosthesis device may be in the compressed state (i.e., prior to deployment), in the partially expanded state (i.e., during deployment), and/or in the expanded state (i.e., fully deployed).

At 1106, a first anatomical structure in the target image may be automatically detected. The first anatomical structure may be visible in the contrast enhanced image, and not visible (or difficult to visualize) without contrast.

Examples of first anatomical structures include: the annulus line, a lower annulus of a subject's native aortic valve, middle of a subject's native aortic valve, top of a subject's native aortic valve, one or more commissures of a subject's native aortic valve, the leaflets of a subject's native aortic valve, the sinuses next to a subject's native aortic valve.

The first anatomical structure may be automatically detected by feeding the target image into a machine leaning model trained on a training dataset of sample images labeled with a ground truth indicating the first anatomical structure.

Alternatively, or additionally, the first anatomical structure may be automatically detected by processing the target image by cross correlation the target image with the 3D image, and mapping the first anatomical structure found in the 3D image to the target image. A projection of the 3D image to a 2D plane is computed, were the 2D projection of the 3D image is registered with the 2D target image. The projection may be computed, for example, using the pose of the image sensor that captured the 2D image. The pose of the image sensor may be found, for example, using a pose sensor, and/or by optical character recognition to extract text of the target image indicating the pose of the image sensor. The first anatomical structure may be found on the 2D projection, and mapped to the target image using the registration.

At 1108, optionally, another image (e.g., 2D fluoroscopic image) is obtained when no contrast was injected (also referred to herein as a non-contrast image), such as after the injected contrast has dissipated.

At 1110, a second anatomical structure associated with the native valve may be automatically detected in the non-contrast image. The second anatomical structure, which cannot be visualized and/or is difficult to visualize in the image with contrast. The second anatomical structure may be visible and/or better visualized in images without contrast.

The second anatomical structure may be located within the native valve of the subject.

Examples of second anatomical structures include: calcification signature (i.e., pattern of calcification deposits), tissue thickening, and blood flow derived dynamic changes.

At 1112, a fiducial object, located in both the non-contrast image(s) and the contrast image(s), may be automatically detected.

The fiducial object may be visible in the non-contrast image(s) and in the contrast image(s).

Optionally, the fiducial object is a catheter located in the aorta of the subject, for example, the catheter used to administer the contrast (e.g., pigtail).

At 1114, the first anatomical structure may be automatically co-located to the second anatomical structure. The automatic co-locating of the first anatomical structure to the second anatomical structure may be based on an identified spatial relationship between the first and second anatomical structures.

The first anatomical structure may be detected without requiring presence of the fiducial object in the image. The first anatomical structure may be detected using the combined mapping, which was computed using the presence of the fiducial object. The fiducial object may be moved and/or removed after the combined mapping has been computed.

Optionally, the co-locating is done by computing a first sub-mapping between the first anatomical structure and the fiducial object. The sub-mapping may be, for example, a spatial relationship, one or more distances (e.g., in pixels and/or millimeters), one or more vectors that indicate relative direction, a geometric shape of a defined size (e.g., triangle having a vertex at the center of mass and a base of the first anatomical structure), and/or a mapping function. A second sub-mapping between the second anatomical structure and the fiducial object depicted may be computed. A combined mapping between the first structure and the second structure may be computed according to a combination of the first sub-mapping and the second sub-mapping.

Optionally, the first mapping and the second mapping are computed for a pair of the non-contrast image and the contrast enhanced image captured at a same pose of an imaging device, same subject orientation, and same zoom.

Alternatively or additionally, the spatial relationship between the first and second anatomical structures may be calculated from the 3D image (e.g., CT or MRI) of the subject taken, optionally take prior to the intervention, for example, as described herein.

At 1116, subsequent non-contrast images may be obtained.

At 1118, the first anatomical structure, which is difficult to visualize and/or cannot be visualized on non-contrast images, may be detected on the non-contrast image(s).

The first anatomical structure may be detected by applying the co-location to the second anatomical structure, which may be automatically detected on the non-contrast images. For example, the calcification signature is automatically detected on the non-contrast image, while the annulus line cannot be visualized on the non-contrast image.

The combined mapping may be applied to the detected second anatomical structure to detect the location of the first anatomical structure on the non-contrast image.

In an exemplary implementation, the first structure is a contour of an annulus of the native valve, the second structure is calcium deposits on the valve, and the fiducial object is a catheter (e.g., pigtail) located in the aorta.

Alternatively or additionally, the location of the first anatomical structure on the non-contrast image is obtained as an outcome of feeding a combination of the non-contrast image(s) and the contrast image(s) into the machine learning model. The machine learning model may be trained on a training dataset of multiple records. Each record may include a combination of sample non-contrast image(s) that depict the structure associated with the valve (e.g., second structure) and optionally the fiducial object, a corresponding sample contrast image(s) that depicts the first anatomical structure (e.g., annulus line) and optionally the fiducial object, and a ground truth indicating of the first anatomical structure on the non-contrast image(s).

The ML model may be dynamically trained using a dynamically created training dataset for the specific patient, where the ground truth is computed using the co-location, such as the combined mapping. Alternatively or additionally, the ML model is previously trained using images from different subjects.

At 1120, the location of the annulus line (or other first anatomical structure) may be dynamically tracked on subsequent images without contrast. The tracking may be done, by mapping the location of the annulus line detected on a baseline image without contrast (e.g., computed as described with reference to 1112) to a subsequent image without contrast, based on a registration between the baseline image and subsequent image. For example, computing optical flow between the baseline image and the subsequent image, and applying the optical flow to map the annulus line from the baseline image to the subsequent image. The mapping from the baseline image to the subsequent image may be computationally efficient in comparison to re-computing the combined mapping for each new image, which may enable real-time tracking.

Alternatively or additionally, pairs of sequentially obtained non-contrast images are fed to a machine learning model for tracking a location of the annulus line. The pairs of images may include the initial image with identified annulus line, and subsequent image without identified annulus line, both of which may be non-contrast images. The ML model may be the dynamically trained ML model, and/or another ML model trained on a training dataset of records, where a record includes a pair of a sample initial image with detected annulus and a subsequent image without detected annulus, and the ground truth is the subsequent image with marked detected annulus.

At 1122, the co-located first anatomical structure (e.g., annulus line) may be presented on a display, for example, as a visual overlay over the target image without contrast.

At 1124, a change in settings may be detected, for example, a change in at least one of the zoom, subject orientation, and the pose of the imaging device, may be detected.

The detected change may trigger a re-computation of the combined mapping (e.g., as in 1114). The new combined mapping may be used for subsequent images at the changed settings.

Alternatively or additionally, the combined mappings may be adapted according to the detected change, for example, by computing a transformation mapping to apply to the combined mappings where the transformation mapping is from the previous settings to the new settings.

Alternatively or additionally, detected change is fed to the machine learning model in combination with the non-contrast image.

At 1126, one or more features described with reference to 1104-1124 are iterated while the valve prosthesis device is transported on its way to be implanted in a native valve and/or while the valve prosthesis device is being positioned and/or deployed.

Referring now back to FIG. 10, features described with reference to FIG. 10 may be implemented for detecting and/or tracking the annulus line, which may be used for computing the width and/or tilt of the THV relative to the annulus line, as described herein. The features described with reference to FIG. 10 may be implemented with, replaced by, combined with, and/or used in addition to, other features of other methods described herein.

The approach described with reference to FIG. 10 tracks the annulus line on non-contrast 2D image(s) where the annulus line cannot be seen and/or is difficult to detect. The method is described in reference to annulus line tracking but may be used to track other anatomical structures which may be required as part of medical procedure. The method may be used for other procedures which require tracking of other anatomical structures which are not visible or not sufficiently visible on non-contrast images.

At 1402, a 3D image of the subject may be accessed. The 3D image may be captured pre-procedure, for example, a CT and/or MRI scan. The 3D image may depict a native valve of the subject for transcatheter replacement in the subject, and/or other anatomical structures which require tracking in other procedures, optionally where the anatomical structures are not visible or not sufficiently visible in non-contrast images.

The native valve of the subject may be, for example, a native aortic valve, a tricuspid valve, and a pulmonary valve.

The subject may be prepared for performance of the medical intervention, for example, a transcatheter aortic valve replacement procedure.

The subject may be undergoing a medical intervention, such as a transcatheter valve replacement procedure for replacement of the native valve.

At 1404, anatomical features are identified on the 3D image. Exemplary anatomical features include calcification deposits, and/or an annulus plane. The annulus plane may be computed by detecting three nadirs of the three cusps of the aortic valve. The annulus plane intersects the three nadirs.

The pattern of calcification deposits may be referred to herein as a calcium signature. The calcium may be deposited in multiple clumps, which may be “randomly” distributed, in the sense that each patient is expected to have their own unique pattern of distribution of calcium deposits.

The calcium deposits in the 3D image may be projected to a 2D plane which represents a template. The template may be matched to 2D images captured during the procedure, for example, in real time or near real time. The template may be of a pattern of multiple calcium deposits, within the leaflets, annulus, native valve, heart muscle, aortic wall, and/or other nearby tissues. The template may include tissues that are not impacted by an event, such as inflation of a balloon within the native valve (e.g., valvuloplasty), to enable performing the registration after the event.

The calcification deposits may be detected, for example, according to a voxels having Hounsfield values within a range that defines calcium.

The pre-procedure image may be used for generating a template which may be registered (e.g., substantially matched and/or correlated) with a signature of a 2D image (e.g., fluoroscopic image) obtained during the procedure. The registration between the 2D image obtained during the procedure (e.g., in real time or near real time) and the 3D image (e.g., obtained pre-procedure) enables mapping anatomical features found in the 3D image to the 2D image, even when the anatomical features are not visible or not sufficiently visible on the 2D image.

At 1406, a 2D target image is obtained during the procedure, optionally in real time or near real time. For example, a 2D image depicting a native valve of a subject. The 2D target image may be non-contrast. The annulus line (or other structure) being detected and/or tracked is not visible and/or not visible well on the non-contrast image.

At 1408, the calcification signature in the 2D target image is detected, for example, by thresholding pixel intensity values to values indicating calcium, machine learning approaches, edge detection, and the like. In other embodiments, other anatomical features are identified on the 2D image.

Optionally, the calcification signature is identified on an anatomical structure(s) that shares similar displacement during cardiac and/or breathing cycles with the annulus line.

Optionally, the calcification signature is identified in tissues that are not significantly impacted by a balloon inflated within the native valve. For example, inflation of the balloon displaces the calcification of the leaflets. This allows using the same calcification signature before and after inflation of the balloon.

At 1410, a calcification template is selected from multiple calcification templates of a 3D image of the subject according to a pose of the image sensor that captured the 2D target image.

Optionally, the calcification template is computed by projecting the 3D image with the annulus plane to a 2D plane having the pose of the image sensor.

Alternatively or additionally, the calcification template is computed by searching the calcification signature for a 2D calcification template of the 3D image according to a correlation requirement. Optionally, multiple 2D projections of the 3D image are pre-computed according to predicted poses of the image sensor. The search may be done using the pre-computed 2D projections, to find the closest matching projection. For example, the 2D projection with highest correlation to the calcification signature of the 2D target image.

The annulus plane (or other structure in 3D) is mapped to an annulus line (or 2D projection of the 3D structure) on the calcification template. For example, the annulus plane in the 3D image is projected to a 2D annulus line during the projection of the 3D image to the 2D plane of the calcification template.

At 1412, the 2D target image may be registered to the calcification template. Registration may be performed, for example, by matching features, and/or other approaches for image registration.

At 1414, the annulus line (or other structure) depicted in the calcification template may be mapped to the registered 2D target image.

At 1416, the annulus line is provided, optionally presented on the 2D target image such as an overlay.

At 1418, one or more features described with reference to 1404-1416 may be iterated. For example, for iteratively tracking the annulus line on subsequent images. Alternatively or additionally, the annulus line may be tracked on subsequent non-contrast 2D image by dynamically correcting for the pose of the image sensor. For example, computing a transformation between the previous pose of the image sensor and the new pose of the image sensor, and applying the transformation to the annulus line of the previous image to obtain the new location of the annulus line on the new image.

The descriptions of the various embodiments of the present invention have been described in reference to a trans-catheter aortic valve replacement intervention; however, the embodiments of the present invention are not limited to such intervention and may include any trans-catheter valve replacement intervention; e.g., tricuspid valve, mitral valve and pulmonary valve replacement intervention.

As used herein, the term aortic valve prosthesis device represents a not necessarily limiting and/or exemplary implementation. Other medical devices (e.g., other valve prosthesis device) which are inserted via a trans-catheter procedure into lumen of the body may be used.

The descriptions of the various embodiments of the present invention have been described in reference to template of calcium signature of the subject; this is a not necessarily limiting and/or exemplary implementation. Other anatomical structure (e.g., specific-subject anatomical structures) may be used, for example: tissue thickening and/or blood flow derived dynamic changes.

The descriptions of the various embodiments of the present invention have been described in reference to obtaining an indication of the annulus line of the heart of the subject; this is a not necessarily limiting and/or exemplary implementation. Other structures within a native valve of a subject may be used as well; for example: middle of a subject's native aortic valve, top of a subject's native aortic valve, one or more commissures of a subject's native aortic valve, the leaflets of a subject's native aortic valve and the sinuses next to a subject's native aortic valve.

The descriptions of the various embodiments of the present invention have been described in reference to CT or MRI images taken prior to the procedure; however, the embodiments of the present invention are not limited to such imaging techniques and may include any imaging techniques; e.g., angiography, ultrasound and Doppler.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant ML models will be developed and the scope of the term ML model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-54. (canceled)

55. A computer implemented method of guiding a trans-catheter aortic valve replacement intervention in a subject, comprising:
in a plurality of iterations during dynamic expansion and/or dynamic adaption of a pose of an aortic valve prosthesis device in an aorta of the subject:
accessing at least one fluoroscopic image depicting the aortic valve prosthesis device in the aorta of the subject, the at least one fluoroscopic image excludes injected contrast,
wherein the at least one fluoroscopic image is a two dimensional (2D) image;
analyzing the at least one fluoroscopic image to identify a distal end of the aortic valve prosthesis device; and
dynamically computing a device depth of the aortic valve prosthesis device relative to an anatomical landmark,
wherein anatomical landmark is difficult to directly visualize or cannot be directly visualized on the at least one fluoroscopic image that excludes injected contrast.

56. The computer implemented method of claim 55, further comprising generating an overlay over the at least one fluoroscopic image indicating a first line indicating the anatomical landmark, a second line indicating the aortic valve prosthesis device, wherein the device depth is computed as distance between the first line and the second line.

57. The computer implemented method of claim 55, wherein presenting comprises generating a graphical user interface (GUI) indicating a target range suitable for deployment of the aortic valve prosthesis device, and regions external to the target range indicating intensity of closeness to the target range, and a visual indication of the device depth within the target range or the regions external to the target range.

58. The computer implemented method of claim 55, wherein analyzing comprises feeding the at least one fluoroscopic image into a machine learning model trained for segmenting the aortic valve prosthesis device within the at least one fluoroscopic image.

59. The computer implemented method of claim 58, wherein the machine learning model is trained on a training dataset of a plurality of sample fluoroscopic images depicting sample aortic valve prosthesis devices, labelled with a ground truth segmentation of the sample aortic valve prosthesis device.

60. The computer implemented method of claim 55, further comprising monitoring for deviation of the device depth from a target range.

61. The computer implemented method of claim 55, wherein the anatomical landmark comprises an annulus line of the aortic valve.

62. The computer implemented method of claim 55, wherein the device depth is computed from a distal end location of the aortic valve prosthesis device to the anatomical landmark.

63. The computer implemented method of claim 62, further comprising determining at least one of a maximal distance and minimal distance of the distal end location of the aortic prosthesis device relative to the anatomical landmark based on historical values.

64. The computer implemented method of claim 55, wherein the device depth is computed from a proximal end location of the aortic valve prosthesis device to the anatomical landmark.

65. The computer implemented method of claim 64, wherein the anatomical landmark comprises at least one of: coronary orifices and annulus line of the aortic valve.

66. The computer implemented method of claim 55, further comprising presenting the device depth on a display.

67. An apparatus for guiding a trans-catheter aortic valve replacement intervention in a subject, comprising:
at least one processor executing a code for:
in a plurality of iterations during dynamic expansion and/or dynamic adaption of a pose of an aortic valve prosthesis device in an aorta of the subject:
accessing at least one fluoroscopic image depicting the aortic valve prosthesis device in the aorta of the subject, the at least one fluoroscopic image excludes injected contrast,
wherein the at least one fluoroscopic image is a two dimensional (2D) image;
analyzing the at least one fluoroscopic image to identify a distal end of the aortic valve prosthesis device; and
dynamically computing a device depth of the aortic valve prosthesis device relative to an anatomical landmark,
wherein anatomical landmark is difficult to directly visualize or cannot be directly visualized on the at least one fluoroscopic image that excludes injected contrast.

68. The apparatus of claim 67, further comprising code for generating an overlay over the at least one fluoroscopic image indicating a first line indicating the anatomical landmark, a second line indicating the aortic valve prosthesis device, wherein the device depth is computed as distance between the first line and the second line.

69. The apparatus of any one of claim 67, wherein presenting comprises generating a graphical user interface (GUI) indicating a target range suitable for deployment of the aortic valve prosthesis device, and regions external to the target range indicating intensity of closeness to the target range, and a visual indication of the device depth within the target range or the regions external to the target range.

70. The apparatus of claim 67, wherein analyzing comprises feeding the at least one fluoroscopic image into a machine learning model trained for segmenting the aortic valve prosthesis device within the at least one fluoroscopic image.

71. The apparatus of claim 70, wherein the machine learning model is trained on a training dataset of a plurality of sample fluoroscopic images depicting sample aortic valve prosthesis devices, labelled with a ground truth segmentation of the sample aortic valve prosthesis device.

72. The apparatus of claim 67, further comprising code for monitoring for deviation of the device depth from a target range.

73. The apparatus of claim 67, wherein the anatomical landmark comprises an annulus line of the aortic valve.

74. The apparatus of claim 67, wherein the device depth is computed from a distal end location of the aortic valve prosthesis device to the anatomical landmark.

75. The apparatus of claim 74, further comprising code for determining at least one of a maximal distance and minimal distance of the distal end location of the aortic prosthesis device relative to the anatomical landmark based on historical values.

76. The apparatus of claim 67, wherein the device depth is computed from a proximal end location of the aortic valve prosthesis device to the anatomical landmark.

77. The apparatus of claim 76, wherein the anatomical landmark comprises at least one of: coronary orifices and annulus line of the aortic valve.

78. The apparatus of claim 67, further comprising code for presenting the device depth on a display.

79-96. (canceled)

97. The computer implemented method of claim 55, wherein the accessing, the analyzing, and the dynamically computing, are performed when the aortic valve prosthesis device is partially expanded.
